(19) United States
(12) Patent Application Publication
(10) Pub. No.: US 2015/0023954 A1
(43) Pub. Date: Jan. 22, 2015

(54) POTENTIATING ANTIBODY-INDUCED COMPLEMENT-MEDIATED CYTOTOXICITY VIA PI3K INHIBITION

(71) Applicant: MEMORIAL SLOAN-KETTERING CANCER CENTER, New York, NY (US)

(72) Inventors: Xiaohong Wu, Forest Hills, NY (US); Wolfgang W. Scholz, San Diego, CA (US); Govind Ragupathi, New York, NY (US); Philip O. Livingston, Bluffton, SC (US)

(21) Appl. No.: 14/387,153

(22) PCT Filed: Mar. 14, 2013
(86) PCT No.: PCT/US13/31278

§ 371 Date: Sep. 22, 2014

Related U.S. Application Data
(60) Provisional application No. 61/614,942, filed on Mar. 23, 2012.

(51) Int. Cl. A61K 39/395; A61K 39/00; A61K 39/385; C12Q 1/68; A61K 31/5377; A61K 39/39; G01N 33/574; A61K 31/4745; A61K 31/4375

(52) U.S. Cl. CPC A61K 39/39558; A61K 31/4745; A61K 39/0011; A61K 39/385; A61K 31/4375; A61K 31/5377; A61K 39/39; G01N 33/57484; C12Q 1/6886; A61K 2039/505; C12Q 2600/16; C12Q 2600/106; A61K 2039/545
USPC 424/133.1; 424/277.1; 424/193.1; 424/174.1; 436/501; 435/7.1; 506/9; 435/7.92; 435/6.12; 436/94

(57) ABSTRACT

Methodologies and technologies for potentiating antibody-based cancer treatments by increasing complement-mediated cell cytotoxicity are disclosed. Further provided are methodologies and technologies for overcoming ineffective treatments correlated with and/or caused by sub-lytic levels of complement-activating monoclonal antibodies (“mAb”) against cancer antigens or cancer antigens with low tumor cell density. While detectable levels of passively administered or vaccine-induced mAb against some antigens are able to delay or prevent tumor growth, low levels of mAb induce sublytic levels of complement activation and accelerate tumor growth. This complement-mediated accelerated tumor growth initiated by low mAb levels results in activation of the PI3K/AKT survival pathway. Methodologies and technologies relating to administration of PI3K inhibitors to overcome low dose mAb-initiated, complement-mediated PI3K activation and accelerated tumor growth are disclosed.

### CROSS-REFERENCE TO RELATED APPLICATIONS

This application claims priority to U.S. provisional application Ser. No. 61/614,942, filed Mar. 23, 2012, the entirety of which is hereby incorporated herein by reference.

### BACKGROUND

Monoclonal antibodies (“mAb”) are widely used in cancer therapy. They are utilized in a variety of ways, including diagnosis, monitoring, and treatment of disease. When used therapeutically, monoclonal antibodies achieve their effects through various mechanisms. For example, some block growth factor receptors, effectively arresting proliferation of tumor cells. Alternatively or additionally, some monoclonal antibodies recruit cytotoxic effector cells such as monocytes and macrophages through a process known as antibody-dependent cell mediated cytotoxicity (“ADCC”). Some monoclonal antibodies bind complement, leading to direct cell death in a process known as complement dependent cytotoxicity (“CDC”).

The complement system is an enzyme cascade comprising a collection of blood and cell surface proteins that assist antibodies in clearing pathogens from an organism. The complement system comprises approximately 30 different proteins, including serum proteins, serosal proteins, and cell membrane receptors. Some complement proteins bind to immunoglobulins or to membrane components of cells. Others are proenzymes that, when activated, cleave one or more other complement proteins and initiate an amplifying cascade of further cleavages. The end-result of this cascade is massive amplification of the response and activation of the cell-killing membrane attack complex. The complement system has four major functions, including lysis of infectious organisms, activation of inflammation, opsonization and immune clearance.

Three different complement pathways have been defined: the classical complement pathway, the alternative complement pathway, and the mannose-binding lectin pathway. The classical pathway is activated following binding of monoclonal antibodies (“mAbs”) to tumor cells. It is initiated by binding of the C1 complex to mAbs in close proximity to the tumor cell membranes. Complement activation on the cell surface results in formation of the membrane-bound C3 and C5-convertases, which are enzyme complexes that cleave and activate C3 and C5, respectively. The cleavage of C3 results in the generation of C3b, which becomes covalently bound to the cell surface. Once bound at the cell surface, C3b amplifies the complement cascade. As complement activation is tightly regulated (even in tumor cells), C3b is rapidly degraded into peptide fragments iC3b and C3dg. These fragments remain cell-bound and function to promote complement receptor-enhanced antibody-dependent ADCC to binding on CR3 on leukocytes. The lectin and alternative pathways are generally activated by pathogens. All three pathways merge at C3, which is then converted into C3a and C3b. The further formed C5 convertase from C3b cleaves C5 into C5a and C5b. C5b with C6, C7, C8, and C9 complex to form the membrane attack complex (MAC), which is inserted into the cell membrane, forms a hole in the membrane, and initiates cells lysis.

Complement-activating monoclonal antibodies have been extensively utilized for the treatment of patients with tumors of different histotypes. Nonetheless, the overall importance of complement activation to the efficacy of mAb-based cancer therapies remains under investigation. Clinically approved mouse anti-epithelial cell adhesion molecule and humanized anti-CD54 activate complement in vitro and medicate ADCC. mAbs directed against HER2 and epithelial growth factor receptor 1 also activate complement in vitro. Chimeric and mouse mAb against CD20 mediate tumoricidal effects in vivo through both ADCC and CDC. However, the primary mechanism of action of other anti-tumor mAbs does not appear to involve complement.

It has been postulated that the lytic potential of complement activation by anti-cancer mAbs may be inhibited by membrane-bound complement regulatory proteins (mCRP). The level of complement activation on cell membranes is regulated by the expression of mCRP, which evolved to protect normal cells from uncontrolled complement-mediated injury. mCRP comprise complement receptor 1 (CD35), membrane cofactor protein (CD46), decay-accelerating factor (CD55), and homologous restriction factor 20 (CD59). CD35, CD46, and CD55 inhibit the deposition of C3 fragments on the cell surface and thereby limit complement-dependent cellular cytotoxicity. CD59 prevents the formation of membrane attack complexes and the subsequent osmotic lysis of the target cell. Over-expression of these mCRP on tumor cells may prevent efficient complement-activation by anti-cancer antibodies.

### SUMMARY

Embodiments of the invention result from the surprising discovery that while high levels of anti-tumor antibodies have the ability to activate the complement cascade, low levels of anti-tumor antibodies can, in fact, induce sublytic levels of complement activation and accelerate tumor growth. For example, although an anti-tumor, complement-activating mAb may be administered at a sufficient dose to initially cause CDC or ADCC of the targeted cancer cell, in vivo levels of the mAb decrease over time. Thus, ironically, a therapeutically effective dose will eventually result in a low dose capable of propagating survival and growth of remaining cells (i.e., sublytic complement activation). This counter-intuitive high/low dichotomy is mediated by the phosphatidylinositol 3-kinase (“PI3K”) cell survival pathway. The present invention further discloses that pharmacological inhibition of the PI3K pathway sensitizes cells to CDC mediated by anti-tumor antibodies. Pharmacological inhibition of the PI3K pathway not only prevents accelerated tumor growth mediated by low levels or doses of anti-tumor antibodies (i.e., sublytic complement activation), it can also potentiate the therapeutic efficacy of standard high doses of anti-tumor monoclonal antibodies and cancer vaccine-induced antibodies. Thus, in some embodiments of the invention, a specific or non-specific PI3K inhibitor is concurrently administered with a complement-activating mAb to increase the effectiveness of mAb-based cancer treatments and reduce the ability of mAbs to perpetuate survival of cancer cells as levels of the antibody decrease following administration.

In an embodiment of the invention, there is provided a method of potentiating an antibody-based cancer treatment. The method comprises administering to a subject a therapeutically effective amount of at least one complement-mediating antibody against a cancer antigen, or a cancer vaccine capable of inducing antibodies against the cancer antigen, and concurrently administering to the subject at least one PI3K inhibitor that inhibits one or more components of the PI3K pathway.

In some embodiments, the cancer antigen is selected from the group consisting of GM2, GD2, GD3, fucosyl GM1, Neu5Gc, CD20, Lewis Y, sialyl Lewis A, Globo H, Thomsen-Friedenreich antigen, Tn, sialylated Tn, Mucin 1, adenocarcinoma-associated antigen, prostate-specific antigen, polysialic acid, and CA125. In some embodiments, the complement-mediating antibody is selected from a group consisting of alemtuzumab, bevacizumab, cetuximab, panitumumab, rituximab, pertuzumab, tositumomab, gemtuzumab ozogamicin, and combinations thereof.

In some embodiments, the PI3K inhibitor inhibits Akt1, Akt 2 or Akt3. In certain embodiments, the PI3K inhibitor inhibits p110. In some embodiments, the PI3K inhibitor inhibits p110α. In some embodiments, the PI3K inhibitor inhibits mTOR. In particular embodiments, the PI3K inhibitor is BEZ235. In some embodiments, the PI3K inhibitor is selected from a group consisting of Wortmannin, F-1126, BEZ-235, BKM120, BYL719, XL-147, GDC-0941, BGT226, GSK1059615, GSK690693, XL-765, PX866, GDC0941, CAL101, Perifosine, VQD002, MK2206, and combinations thereof.

Some embodiments of the invention further comprise concurrent administration of at least one MEK inhibitor. Other embodiments comprise administration of PI3K inhibitors without affecting MEK pathways.

In some embodiments of the invention, the therapeutically effective amount of complement-mediating antibody comprises at least one dose of about 1-150 milligrams per kilogram (kg) of body weight of the subject. In some embodiments, the step of administering an anti-tumor antibody comprises administering at least one dose of about 40-50 milligrams per kilogram of body weight to the subject. In certain embodiments, the PI3K inhibitor is orally or parenterally administered in an amount sufficient to deliver from about 1-150 milligram per kilogram (kg) of body weight of the subject.

In some embodiments, the antibody-based cancer treatment is used for treating a neuroblastoma, lymphoma, colon cancer, breast cancer, sarcoma, melanoma, pancreatic cancer, prostate cancer, ovarian cancer or small cell lung carcinoma.

Some embodiments of the invention further comprise determining a level of expression of the tumor cell surface antigen and treating a subject based in part on the level of the antigen. Some embodiments of the invention further comprise concurrent administration of an anti-cancer treatment. In particular embodiments, the anti-cancer treatment is selected from the group consisting of cytotoxic agents, radiation, and surgery. In certain embodiments, the cytotoxic agents are selected from the group consisting of cisplatin, carboplatin, doxorubicin, etoposide, cyclophosphamide, methotrexate, taxol, Gemcitabine and celecoxib.

In some embodiments of the invention, methods are provided for administering a cancer vaccine to a subject. The methods comprise concurrently administering a PI3K inhibitor to the subject. In some embodiments, the cancer vaccine is a polyvalent vaccine. In some embodiments, the cancer vaccine is a monovalent vaccine.

In some embodiments, the cancer vaccine induces complement-mediating antibodies against a cell surface antigen selected from the group consisting of a carbohydrate epitope, a glycolipid epitope, a glycoprotein epitope or a mucin. In particular embodiments, the carbohydrate epitope is selected from the group consisting of GM2, GD2, GD3, fucosyl GM1, Neu5Gc, CD20, Lewis Y, sialyl Lewis A, Globo H, Thomsen-Friedenreich antigen, Tn, sialylated Tn, Mucin 1, adenocarcinoma-associated antigen, prostate-specific antigen, polysialic acid, and CA125.

In some embodiments, the cancer vaccine comprises an antigen chemically conjugated to a carrier molecule. In particular embodiments, the carrier molecule is selected from the group comprising keyhole limpet hemocyanin, Neisseria meningitidis outer membrane proteins, multiple antigenic peptide, cationized bovine serum albumin and polylysine.

In some embodiments, the cancer vaccine further comprises an adjuvant. In particular embodiments, the adjuvant is selected from the group comprising CRL-1005 (polypropylene), CpG ODN 1826 (synthetic bacterial nucleotide), GM-CSF (peptide), MPL-SE (monophosphoryl lipid A), GPI-0100 (hydrolyzed saponin fractions), MoGM-CSF (Fc-GM-CSF fusion protein), PG-026 (Peptidoglycan), QS-21 (saponin fraction), synthetic QS-21 analogs, and TiterMax Gold (CRL-8300 (polyoxypropylene; polyoxyethylene).

In another embodiment of the invention there is provided a method for identifying and/or treating subjects suitable for treatment with complement-mediating anti-tumor antibodies. The method comprises quantifying in a sample from a subject suffering from, or susceptible to, cancer an expression level of an antigen that is differentially expressed in cancer cells relative to normal cells, which antigen is recognized by at least one antibody that activates complement; and determining that the expression level is above or below a threshold correlated with responsiveness to complement-activating therapy.

### DEFINITIONS

Anti-tumor antibody: As used herein, the terms “anti-tumor antibody” or “anti-cancer antibody”, which may be used interchangeably, refer to any antibody that is specific to an antigen commonly associated with a cancerous cell or tumor mass. In some embodiments, and antigen is “commonly associated with a cancerous cell or tumor mass” if its presence, level (e.g., above or below a defined threshold amount) and/or activity correlates with a cancerous state. Anti-tumor antibodies according to embodiments of the invention may be polyclonal or monoclonal. They may be human, mouse, chimeric or humanized. Antigens to which anti-tumor antibodies bind may be expressed on the surface of a cancer cell or retained within a local cancer milieu. Anti-tumor antibodies may be directed against an antigen commonly associated with a solid tumor, lymphoma, leukemia, myeloma, etc. In some embodiments, anti-tumor antibodies eradicate free tumor cells and micrometastases. In certain embodiments, anti-tumor antibodies are specific for glycolipids or glycoproteins expressed on the surface of certain cancerous cells; e.g., anti-GM2 antibody, anti-GD2 antibody, anti-sLe^{a} antibody or anti-GD3 antibody. In some embodiments of the invention, anti-tumor antibodies are passively administered. In some embodiments, the anti-tumor antibodies are 3F8, 5B1, R24, PGNX and/or Rituxan. In some embodiments, anti-tumor antibodies include alemtuzumab (Campath), bevacizumab (Avastin®, Genentech); cetuximab (Erbitux®, Imclone), panitumumab (Vectibix®, Amgen), rituximab (Rituxan®, Genentech/Biogen Idec), pertuzumab (Omnitarg®, Genentech), tositumomab (Bexxar, Corixia), and the antibody drug conjugate, gemtuzumab ozogamicin (Mylotarg®, Wyeth). Anti-tumor antibodies may also include Zamly™, epratuzumab, Cotara™, edrecolomab, mitomomab, tositumomab (Bexxar®) CeaVac™, ibritumomab (Zevalin™) and OvaRex (Zevalin®). In some embodiments, anti-tumor antibodies are induced within a subject by administration of anti-cancer vaccine; i.e., vaccine-induced anti-tumor antibodies. In some embodiments, anti-tumor antibodies are conjugated to a payload (e.g., a diagnostic or therapeutic payload). In some particular such embodiments, the payload is or comprises radioactive particles, cytotoxic drugs and/or immunotoxins. In addition to the cytotoxic agents described below, exemplary payloads in particular embodiments of the invention include calicheamicin, maytansinoids and auristatins.

Antagonist: As used herein, the term “antagonist” refers to an agent that i) inhibits, decreases or reduces one or more effects of another agent, for example that block a receptor/agonist interaction; and/or ii) inhibits, decreases, reduces, or delays one or more biological events, for example, inhibit activation of one or more receptors or stimulation of one or more biological pathways. In particular embodiments, an antagonist inhibits activation and/or activity of one or more components of the PI3K pathway (e.g. p110 or Akt). Antagonists may be or include agents of any chemical class including, for example, small molecules, polypeptides, nucleic acids (e.g., RNAi, small interfering RNA, micro RNA), carbohydrates, lipids, metals, and/or any other entity that shows the relevant inhibitory activity. An antagonist may be direct (in which case it exerts its influence directly upon the receptor) or indirect (in which case it exerts its influence by other than binding to the receptor; e.g., binding to a receptor agonist, altering expression or translation of the receptor; altering signal transduction pathways that are directly activated by the receptor, altering expression, translation or activity of an agonist of the receptor).

Antibody polypeptide: As used herein, the terms “antibody polypeptide” or “antibody”, which may be used interchangeably, and in accordance with “anti-tumor antibodies”, refer to polypeptide that specifically binds to an epitope or antigen. In some embodiments, antibody polypeptide is polypeptide whose amino acid sequence includes elements characteristic of an antibody-binding region (e.g., an antibody light chain or variable region or one or more complementarity determining regions (“CDRs”) thereof, or an antibody heavy chain or variable region or one more CDRs thereof, optionally in presence of one or more framework regions). In some embodiments, an antibody polypeptide is or comprises a full-length antibody. In some embodiments, an antibody polypeptide is less than full-length but includes at least one binding site (comprising at least one, and preferably at least two sequences with structure of known antibody “variable regions”). In some embodiments, the term “antibody polypeptide” encompasses any protein having a binding domain, which is homologous or largely homologous to an immunoglobulin-binding domain. In particular embodiments, an included “antibody polypeptides” encompasses polypeptides having a binding domain that shows at least 99% identity with an immunoglobulin binding domain. In some embodiments, an included “antibody polypeptide” is any protein having a binding domain that shows at least 70%, 80%, 85%, 90%, or 95% identity with an immunoglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source, recombinant production in or with a host system, chemical synthesis, etc., or combinations thereof. An antibody polypeptide may be monoclonal or polyclonal, mono-specific or bi-specific. An antibody polypeptide may be a member of any immunoglobulin class, including any of the human classes: IgG, IgM, IgA, IgD, and IgE. In certain embodiments, an antibody may be a complement-activating antibody. Complement-activating antibodies may trigger or enhance both antibody-dependent cellular cytotoxicity (“ADCC”) (e.g., enhancing binding of phagocytic or cytotoxic effector cells such as granulocytes, natural killer cells, monocytes or macrophages) and complement activation. Antibodies may be modified to improve ADCC or complement recruitment. Antibody polypeptides may be chimeric or humanized mouse monoclonal antibodies. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some embodiments, an antibody polypeptide may be a human antibody. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively or additionally, an antibody fragment may comprise multiple chains that are linked together, for example, by disulfide linkages.

Cancer: The terms “cancer” and “cancerous”, as used herein, refer to or describe a physiological, histological or genetic condition in a subject that is characterized by unregulated cell growth or division. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Cancer antigen: The term “cancer antigen”, as used herein, refers to any molecule (e.g., glycolipids or glycoproteins) expressed on the surface of a cancer cell and against which an anti-tumor antibody may be directed or induced by vaccine. Antibodies against cancer antigens induce CDC and/or ADCC, inflammation and phagocytosis of tumor cells. Non-limiting examples of antigens targeted or utilized in embodiments of the invention include: gangliosides such as GM2, GD2, GD3 and fucosyl GM1; glycolipids such as Lewis Y, sialyl Lewis A and Globo H; mono- or disaccharide antigens O-linked to mucins such as Thomsen-Friedenreich antigen (“TF”), Tn and sialylated Tn; Mucin 1 (“MUC1”); adenocarcinoma-associated antigen (“KSA”); prostate-specific antigen (“PSMA”); polysialic acid, and CA125. In some embodiments of the invention, one or more cancer antigens (e.g., unimolecular multiantigenic constructs such as STn cluster, TN cluster and TF clustered antigens) comprises a cancer vaccine capable of inducing active immunity against the cancer antigen(s). See, generally, Philip Livingston and Govind Ragupathi, Carbohydrate Vaccines Against Cancer, in GENERAL PRINCIPLES OF TUMOR IMMUNOTHERAPY: BASIC AND CLINICAL APPLICATIONS OF TUMOR IMMUNOLOGY 297-317 (Howard L. Kaufman and Jedd D. Wolchok eds., Springer 2007).

Complement-mediated Cytotoxicity: The term “complement-mediated cytotoxicity” refers to cytotoxicity that requires presence and/or activity of at least one component of the complement system. In some embodiments, complement-mediated cytotoxicity requires one or more components of the classical pathway of the complement system; in some embodiments, complement-mediated cytotoxicity requires one or more components of the alternative pathway; in some embodiments, complement-mediated cytotoxicity requires one or more components of the antibody-dependent cellular cytotoxicity (“ADCC”) pathway, which can be enhanced by certain antibodies that activate the complement system (i.e, complement receptor-dependent enhancement of ADCC). Complement function in mAb-mediated cancer immunotherapy has been described previously. (see Gelderman, K. A. et al., TRENDS in Immunol., 2004, 25(3):158-164; incorporated by reference herein.)

Concurrent Administration: As used herein, the term “concurrent administration” or “combination therapy” refers to embodiments wherein two or more therapeutic agents, e.g., a monoclonal anti-tumor antibody and a PI3K inhibitor, are administered using doses and time intervals such that the administered agents are present together within the body, or at a site of action in the body such as within a tumor) over a time interval in not less than de minimis quantities, i.e., they are present together in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc. Accordingly, the therapeutic agents may, but need not be, administered simultaneously, almost simultaneously, or together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the invention agents administered within such time intervals may be considered to be administered at substantially the same time. In certain embodiments of the invention concurrently administered agents are present at effective concentrations within the body over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The de minimis concentration of an agent may be, for example, less than approximately 5% of the concentration that would be required to elicit a particular biological response, e.g., a desired biological response. In some embodiments, concurrent administration entails inhibition of one or more biological pathways in addition to the PI3K pathway. For example, a PI3K inhibitor may be concurrently administered with an anti-tumor mAb and an inhibitor of the Ras/Raf/Mek/Erk pathways (e.g., AZD6244 or GSK1120212) and/or a receptor tyrosine kinase inhibitor (e.g., erlotinib).

Cytotoxic agents: The term “cytotoxic agent”, or alternatively “chemotherapeutic agent”, as used herein refers to any molecule or composition of matter used by those of skill in the art of cancer treatment to cause or contribute to cell death (e.g., apoptosis) or to render a cell susceptible to death. Examples of chemotherapeutic agents include any one or more of abarelix, aldesleukin, alemtuzumab, alitretinoin, allopurinol, altretamine, amifostine, anastrozole, arsenic trioxide, asparaginase, axinib, azacitidine, BCG Live, bevacuzimab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, crizotinib, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid, and pharmaceutically acceptable salts, acids or derivatives of any of the above.

Dosage form: As used herein, the terms “dosage form” and “unit dosage form” refer to a physically discrete unit of a therapeutic composition to be administered to a subject. Each unit contains a predetermined quantity of active material (e.g., therapeutic agent). In some embodiments, the predetermined quantity is one that has been correlated with a desired therapeutic effect when administered as a dose in a dosing regimen. In some embodiments, a dosage form may be a combined dosage of anti-tumor antibody and PI3K inhibitor. Those of ordinary skill in the art appreciate that the total amount of a therapeutic composition or agent administered to a particular subject is determined by one or more attending physicians and may involve administration of multiple dosage forms.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses, each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome (e.g., activation of complement-mediated cell death), when administered across a population of patients. In some embodiments, a dosing regimen may comprise the sequential administration of an anti-tumor antibody and a PI3K inhibitor. In particular embodiments, the PI3K inhibitor may be administered between 1-24 hrs prior to administration of any anti-tumor antibody. In some embodiments, a PI3K inhibitor may be administered regularly over a period of days or weeks prior to administration of an anti-tumor antibody. In certain embodiments, an anti-tumor antibody is administered prior to administration of a PI3K inhibitor. The anti-tumor antibody may be administered 1-24 hours prior to administration of the PI3K inhibitor. The anti-tumor antibody may also be regularly administered over a period of days or weeks prior to administration of the PI3K inhibitor. In other embodiments, the anti-tumor antibody and the PI3K inhibitor may be co-administered or concurrently administered. In some embodiments, a dosing regimen comprises vaccination against a cancer antigen, the vaccination being capable of inducing active immunity against the cancer antigen. In certain embodiments comprising vaccination, the dosing regimen is administered after cancer surgery and/or chemotherapy (e.g., following administration of one or more of the cytotoxic agents described above).

High Dose: As used herein, the term “high dose” refers to any dose of anti-tumor antibody whose administration is correlated with (or has sufficient titer to) arresting or slowing tumor growth or cancerous cell division, and/or effecting ADCC or CDC of a cancerous cell, either in vivo or in vitro. In some embodiments, a high dose is a dose that results in serologically detectable levels of the antibody. In some embodiments, a high dose is defined as producing an antibody titer between about 1/160 and 1/1280 at least 4 hours from administration. In some embodiments, a high dose is between about 1-150 milligrams of anti-tumor antibody per kilogram (kg) of body weight of the subject. In some embodiments, a high dose is between about 15-150 milligrams of anti-tumor antibody per kilogram (kg) of body weight of the subject. In some embodiments, a high dose is defined by an antibody dose with a concentration of 1-100 μg/ml; e.g., about 5 μg/ml, about 10 μg/ml, about 115 μg/ml, about 20 μg/ml, about 25 μg/ml, about 30 μg/ml, about 35 μg/ml, about 40 μg/ml, about 45 μg/ml, about 50 μg/ml, or higher. Those of ordinary skill in the art will appreciate that the total amount of a therapeutic composition or agent administered to a particular subject is determined by one or more attending physicians and may involve administration of multiple dosage forms. A “high dose” may also vary depending on the height, weight, sex, age and health of the subject, as well as the severity of disease. A “high dose” may also vary depending on the type of cancer being treated or the particular antibody being administered. A person of skill in the art will be able to account for the subjective variation of a given subject relative to a standard high dose administration.

Low Dose: As used herein, the term “low dose” refers to any dose of an anti-tumor antibody correlated with absence of a therapeutic effect, or with accelerated tumor growth or cancerous cell division in vitro or in vivo. In particular embodiments of the invention, a low dose may be a dose that results in little or no detectable serum antibody within 2-4 hours of dosing. In some embodiments, a low dose is between about 0.01-1.0 milligrams of anti-tumor antibody per kilogram (kg) of body weight of the subject. In some embodiments, a low dose is between about 0.001-1.0 milligram of anti-tumor antibody per kilogram (kg) of body weight of the subject. In some embodiments, a low dose is defined by an antibody dose with a concentration of less than 1.0 μg/m; e.g., about 0.9 μg/ml, about 0.8 μg/ml, about 0.7 μg/ml, about 0.6 μg/ml, about 0.5 μg/ml, about 0.4 μg/ml, about 0.3 μg/ml, about 0.2 μg/ml, about 0.1 μg/ml, about 0.01 μg/ml, about 0.001 μg/ml, about 0.0001 μg/ml or lower. In some embodiments of the invention, a “low dose” is caused by a loss or metabolism of active mAb following administration of a high dose. In other words, as the amount of mAb in the blood or tissue decreases over time following administration, a low dose is effectively created. Thus, ironically, a “high” therapeutically effective dose that mediates ADCC or CDC becomes a “low dose” that propagates survival of the remaining cells. Those of ordinary skill in the art appreciate that the total amount of a therapeutic composition or agent administered to a particular subject is determined by one or more attending physicians and may involve administration of multiple dosage forms. A “low dose” may also vary depending on the height, weight, sex, age and health of the subject, as well as the severity of disease. A “low dose” may also vary depending on the type of cancer being treated or the particular antibody being administered. A person of skill in the art will be able to account for the subjective variation of a given subject relative to a standard low dose administration.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

PI3K Inhibitor: As used herein, the terms “PI3K inhibitor” and “PI3K inhibition” refer to any molecule, entity or composition of matter that blocks or diminishes activation of any activator, component, or effector of the phosphatidylinositol 3-kinase pathway. “PI3K inhibitors” may encompass small molecule pharmaceuticals, biologics and inhibitors of transcription or translation of PI3K components (e.g., siRNA, RNAi, or microRNA). Specific examples of PI3K inhibitors include, LY294002, LY49002, SF-1126 (Semafore Pharmaceuticals), BEZ235 (a.k.a. BEZ235) and BKM120 and BYL719 (Novartis), XL-147 (Exelixis, Inc.), GDC-0941 (Plramed and Genentech) and combinations thereof. PI3K inhibitors for use in embodiments of the invention may be specific or non-specific. In some embodiments, multiple PI3K inhibitors may be administered either separately or in combination, before, during and/or after administration of an anti-tumor antibody. In some embodiments, PI3K inhibition is specific in that, within a complex cellular environment, it preferably targets one or more components of the PI3K pathway rather than another biological pathway (e.g., mitogen-activated protein kinase pathways, protein kinase C signaling, NF-κB signaling, TGF-β signaling, Notch signaling, etc.). In other embodiments, PI3K inhibition may be non-specific, meaning that the PI3K inhibitor affects one or more biological pathways other than the PI3K pathway. In some embodiments, a PI3K inhibitor may be a dual inhibitor. In some embodiments, PI3K inhibition is direct inhibition of one or more components of the PI3K pathway; e.g. inhibiting Akt phosphorylation or inhibiting interaction between a PI3K component and a binding partner. In some embodiments, the PI3K inhibitor physically associates with a PI3K pathway component. In some embodiments, such physical association is reversible; in other embodiments, such physical association is irreversible. In some embodiments, PI3K inhibition is indirect, meaning that it involves upregulation or activation of one or more entities that negatively affect or circumvent PI3K activation. For example, an indirect inhibitor may increase the activity of a phosphatase, which dephosphorylates and down-regulates the activity of an Akt substrate; dephosphorylation of an Akt substrate may also remove Akt-induced inhibition of the substrate. Downstream targets of Akt that may be directly or indirectly affected in embodiments of the invention include, for example: Acinus, APS, Androgen Receptor, Arfaptin 2, AS160, ASK1, Ataxin-1, Bad, Bcl-xL, Bim, B-Raf, BRCA1, CACNB2, CaRHSP1, Caspase-9, CBP, CCT2, Cdc25B, CDK2, CENTB1, Chk1, CK1-D, Connexin 43, Cot (Tpls2), CSP, CTNNB1 (b-Catenin), CTNND2 (Catenin d-2), CUGBP1, DLC1, EDC3, EDG-1, eIF4B, eNOS, Estrogen Receptor-a, Ezh2, Ezrin, FANCA, FLNC, FOXA2, FOXG1, FoxO1a, FoxO3a, FoxO4, Gab2, GATA-1, GATA-2, Girdin, GOLGA3, GSK-3a, GSK-3b, H2B, HMOX1, hnRNP A1, hnRNP E1, Htra2, Huntingtin, IKK-a, IP3R1, IRS-1, Kv11.1 iso5, Lamin A/C, Mad1, MDM2, MLK3, METTL1, MST1, mTOR, MYO5A, Myt1, Ndrg2, NFAT90, NMDAR2C, NuaK1, Nur77, p21, p300, Palladin, PDCD4, PDE3A, PDE3B, Peripherin, PFKFB2, PGC-1, PLCg1, PRAS40 (Akt1S1), PRPK, PTP1B, OIK, Rac1, Raf1 (c-Raf), RANBP3, Ron, S6, SEK1 SH3BP4, SH3RF1, Skp2, SKI, SSB, TAL-1, TBC1D4, TERT, TOPBP1, TRF1, TTC3, Tuberin (TSC2), USP8, VCP, WNK1, XIAP, YAP1, YB1, and Zyxin.

Pretreatment: The term “pretreatment” as used herein refers to the administration of a PI3K inhibitor or other cancer therapy prior to administration of an anti-tumor antibody. Pretreated or pretreatment includes subjects who have received a treatment other than an antibody-based cancer treatment within 1 year, 8 months, 6 months, 3 months, 1 month, 3 weeks, 2 weeks, 1 week, 6 days, 5 days, four days, 3 days, 2 days 24 hours or less prior to administration of the antibody-based treatment.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. One may refer to a subject's response or to a tumor's response. In general these concepts are used interchangeably herein. Tumor or subject response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 92(3):205-16, 2000.

Sample: In some embodiments, the term “sample” as used herein refers to a primary sample obtained from a subject, for example including any or all of the following: a cell or cells, a portion of tissue, blood, serum, ascites, urine, saliva, and other body fluids, secretions, or excretions. Alternatively or additionally, in some embodiments, the term “sample” refers to a preparation obtained by processing a primary sample, for example by subjecting the primary sample to one or more separation steps, and/or one or more amplification steps. In some embodiments, such processing steps of copying nucleic acids (e.g., via reverse transcription, polymerase chain reaction, etc., and/or combinations thereof), etc.

Specifically Binds: As used herein, the term “specifically binds” refers to an entity (e.g., antibody polypeptide) that discriminates among possible binding partners present in an environment in favor of a specific partner; e.g., that binds to a target with greater affinity than it binds to a non-target. In some embodiments, specific binding refers to binding for a target that is favored by a factor at least 10, 50, 100, 250, 500 or 1000 times greater than binding for a non-target.

The ability of an antibody to bind a specific epitope can be described by the equilibrium dissociation constant (K_{D}). The equilibrium dissociation constant (K_{D}) as defined herein is the ratio of the dissociation rate (K-off) and the association rate (K-on) of a an antibody to a cancer antigen. It is described by the following formula: K_{D}=K-off/K-on. In some embodiments, antibodies and antibody compositions disclosed herein bind a cancer antigen with an equilibrium dissociation constant (K_{D}) of about 100 nM, about 90 nM, about 80 nM, about 70 nM, about 60 nM, about 50 nM, about 40 nM, about 30 nM, about 20 nM, about 10 nM, about 9 nM, about 8 nM, about 7 nM, about 6 nM, about 5 nM, about 4 nM, about 3 nM, about 2 nM or less, and/or between 2-10 nM. In some embodiments, cancer antigen binding affinity is determined by competition ELISA using the method of Friquet et al., “Measurements of True Affinity Constant in Solution of Antigen-Antibody Complexes by Enzyme-Linked Immunosorbent Assay,” J. Immuno Methods, 305 (1985).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (e.g., cancer) has been diagnosed with and/or exhibits one or more symptoms of the disease, disorder, or condition. A subject suffering from cancer or tumors may be asymptomatic.

Susceptible to: As used herein, the term “susceptible to” refers to having an increased risk for and/or a propensity for (typically based on genetic predisposition, environmental factors, personal history, or combinations thereof) something, i.e., a disease, disorder, or condition, than is observed in the general population. The term encompasses the understanding that an individual “susceptible” for a disease, disorder, or condition may never be diagnosed with the disease, disorder, or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, in some embodiments, a delay in the onset of a particular symptom is considered one form of reducing the frequency of that symptom. The present invention specifically contemplates treatment such that one or more symptoms is/are reduced (and the condition of the subject is thereby “improved”), albeit not completely eliminated.

Therapeutic agent: As used herein, the phrase “therapeutic agent” refers to any agent that has a therapeutic effect and/or elicits a desired biological and/or pharmacological effect, when administered to a subject.

Therapeutically effective amount: As used herein, the term “therapeutically effective amount” refers to an amount of a therapeutic protein (e.g., anti-tumor antibody) or PI3K inhibitor that is correlated with a predetermined beneficial outcome; i.e., that confers a therapeutic effect on the treated subject. The therapeutic effect may be objective (i.e., measurable by some test or marker) or subjective (i.e., subject gives an indication of or feels an effect). In particular, the “therapeutically effective amount” refers to an amount of a therapeutic antibody or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered as part of a therapeutically effective dosing regimen (i.e., a regimen that shows a statistically significant correlation with a positive outcome when administered to a relevant population) that may comprise a plurality of doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism; the duration of the treatment; and like factors as is well known in the medical arts.

Treatment: As used herein, the term “treatment” (also “treat” or “treating”) refers to any administration of a substance (PI3K inhibitor(s) plus complement-mediating antibody) that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DESCRIPTION OF THE DRAWING

FIG. 1 demonstrates cell surface expression of GM2, GD2, and GD3 on CHLA136luc and LAN-1 neuroblastoma cells and on H524 SCLC cells, and CD20 expression on Hs445 and Daudiluc lymphoma cells. Cell lines were stained by immunofluorescence using appropriate antibodies as labeled. The figure shows histograms of relative fluorescence.

FIG. 2 demonstrates in vivo efficacy of PGNX, R24 and 3F8 administration every week for 4 weeks alone or mixed beginning 2 days after IV challenge with 500,000 CHLA136luc cells in SCID mice. FIG. 2A,B: Single mAb doses (5 μg low dose (L) or 50 μg) or mixed mAb doses (3F8, R24, and PGNX, 50 μg each) were injected IP 2 days after IV challenge. 2B, C: Single mAb doses (1 μg low dose (L) or 50 μg were injected IP 2 days after IV challenge. FIG. 2A, C: Comparison of experimental group survival with control group by Kaplan-Meier methodology. FIG. 2B, D: Student t test used for statistical comparison of tumor growth measured by luciferase expression at 6 weeks in experimental groups compared with control mice: increased cell growth (¤ P<0.05) or decreased cell growth (*P<0.05, ** P<0.01).

FIG. 3 demonstrates in vitro cell growth study with a range of doses of monoclonal antibodies on selected cell lines: A. CHLA136Luc cells (neuroblastoma); B. Lan-1-Luc cells (neuroblastoma); C. H524 (SCLC); D. Hs445 (lymphoma); F. Daudi (lymphoma); 20,000 cells were plated in triplicate and treated with human complement and different amounts of antibodies, antibodies alone, or complement alone, as indicated for 24 hours. Cellular proliferation was quantitated using the WST-1 assay. Each bar represents the mean of triplicates. Student t test results for statistical significance are as indicated: increased cell growth with complement plus low mAb levels (¤ P<0.05, ¤¤ P<0.01, ¤¤¤ P<0.001) or decreased cell growth with complement plus higher mAb levels compared to complement (HuC′) alone (* P<0.05, ** P<0.01, *** P<0.001).

FIG. 4 demonstrates correlation between low-dose PGNX induced phosphorylated Akt (p-Akt) expression and phosphorylated PRAS40 (p-PRAS40) expression in CHLA136Luc cell extracts by Western blot analysis. 4A: PGNX dose impact on pAkt expression. 4B: Time course of PGNX 0.001 μg/m impact on p-Akt expression and its downstream substrate P-PRAS40. 4C: Impact of BEZ235 on p-Akt and its downstream substrate P-PRAS40 expression for CHLA136Luc cells treated after treatment with PGNX (0.001 μg/m) for 4 hours.

FIG. 5 demonstrates the impact of treatment for 18 hours with increasing doses of BEZ235 and 3F8 on CHLA136Luc cell growth (FIG. 5A) and BEZ235 and Rituxan on DaudiLuc cell (FIG. 5B) growth in WST-1 assays. All PI3K inhibitor BEZ235 dose levels prevented the low mAb dose (plus complement) growth acceleration and increased higher mAb dose (plus complement) cytotoxicity. FIG. 5 also demonstrates the impact of treatment for 18 hours with increasing doses of AKT inhibitors MK2206 (FIG. 5C) and BKM120 (FIG. 5D) and PGNX on CHLA136Luc cell growth in WST-1 assays. Once again, all AKT inhibitor dose levels prevented the low mAb dose (plus complement) growth acceleration and increased higher mAb dose (plus complement) cytotoxicity. Each bar represents the mean of triplicate testing. P values compared with control cells treated with human complement alone are as indicated: increased cell growth (¤ P<0.5) or decreased cell growth (* P<0.05, ** P<0.01, *** P<0.001).

FIG. 6 demonstrates the impact of BEZ235 on PGNX and/or 3F8 activity in vivo. Mice received BEZ235 25 mg/kg (FIG. 6A, B) or 12.5 mg/kg (FIG. 6C) by gavage beginning 4 days after IV challenge with 500,000 CHLA136Luc cells and continuing daily for 2 weeks. PGNX and/or 3F8 at the indicated doses were injected IV(PGNX) or IP (3F8) starting a day later (5 days after tumor challenge) and re-injected once a week for 4 weeks. 6A,C: Comparison of experimental group survivals to control group by Kaplan-Meier methodology. 6B: Student t test used for statistical comparison of tumor growth measured by luciferase expression at 8 weeks in experimental groups compared with control mice. Results for statistical significance are indicated. As previously demonstrated in vitro, BEZ235 also prevented low dose mAb induced growth acceleration and increased high mAb dose induced growth inhibition in vivo.

FIG. 7 demonstrates the low dose effect of 5B1 mAb upon Colo205 cells in vitro and the impact of BEZ235 administration. FIG. 7A shows complete inhibition of p-AKT expression for cells treated for 4 hrs with BEZ235 at doses of 0.5 μM or higher. FIG. 7B shows complete inhibition of p-Akt expression in cells treated with BEZ235 at 1 μM for 2 hrs or longer. FIG. 7C shows low dose 5B1 (0.001 μg/ml) (plus human complement (HuC)) induced increased p-Akt expression starting after 4 hrs of treatment. Fig. D shows cells treated with 5B1 (0.001 μg/ml; i.e., low dose) and HuC′ (5%) with or without 1 μM BEZ235 for 4 hrs results in increased p-AKT with low dose 5B1 alone, and decreased p-AKT with BEZ235 alone or in combination with low dose 5B1. The bar graph represents ratio of p-AKT versus loading control Actin.

FIG. 8 demonstrates AKT-immunofluorescent staining of Colo205 cells treated with 5B1 at 0.001 μg/ml and human complement (HuC′; 5%) with or without 1 μM BEZ235. Low dose 5B1 alone induced increased cell growth and AKT expression. The combination BEZ235 with low dose 5B1 decreased escalated p-AKT expression as shown by the intensity of p-AKT(green) versus cell threshold area. (graph). Image were taken at 2× magnification.

FIG. 9 demonstrates a cell growth assay of Colo205 cells treated overnight with mAb 5B1 and human complement (HuC; 5%) and increased doses of BEZ235 (FIG. 9A), Wortmannin (FIG. 9B), MK2206 (FIG. 9C) and BKM120 (FIG. 9D). BEZ235 (a PI3K/AKT/mTor inhibitor) at all doses tested enhanced all tested doses of mAb 5B1 cell cytotoxicity (OD415 nm indicating cell survival). Wortmannin (a PI3K/AKT inhibitor) showed similar but less potent effects. MK2206 (a specific allosteric AKT inhibitor) and BKM120 (a specific inhibitor of class 1 PI3K) also enhanced the efficacy of 5B1 cytotoxicity at all doses tested.

### DETAILED DESCRIPTION OF CERTAIN EMBODIMENTS

The present invention addresses a surprising dichotomy that occurs in antibody-based anti-cancer treatments. A variety of monoclonal antibodies (“mAbs”) against cancer antigens are capable of prolonging a disease-free state and overall survival in preclinical studies and in clinical responses when tumors known to be strongly positive for the relevant antigens are targeted. Several such mAbs have been FDA approved for these purposes. Monoclonal antibodies against gangliosides GD2 and GD3 have demonstrated both preclinical efficacy and clinical responses in neuroblastoma and melanoma patients, respectively, again in the setting of strongly antigen-positive tumors. (see, e.g., Houghton A. N., et al “Mouse monoclonal IgG3 antibody detecting GD3 ganglioside: a phase I trial in patients with malignant melanoma”, Proc. Natl. Acad. Sci. U.S.A., 1985, 82(4):1242-6; Imai M., et al. “Complement-mediated mechanisms in anti-GD2 monoclonal antibody therapy of murine metastatic cancer”, Cancer Res., 2005, 65(22):10562-8; Irie R. F., et al. “Human monoclonal antibody to ganglioside GM2 for melanoma treatment”, Lancet, 1989, 1(8641):786-7; Kushner B. H., et al. “Phase II trial of the anti-G(D2) monoclonal antibody 3F8 and granulocyte-macrophage colony-stimulating factor for neuroblastoma”, J. Clin. Oncol., 2001, 19(22):4189-94; Nasi M. L., et al. “Anti-melanoma effects of R24, a monoclonal antibody against GD3 ganglioside”, Melanoma Res., 1997, 7 Suppl 2:S155-62; Retter M. W., et al. “Characterization of a proapoptotic antiganglioside GM2 monoclonal antibody and evaluation of its therapeutic effect on melanoma and small cell lung carcinoma xenografts”, Cancer Res., 2005, 65(14):6425-34; Zhang H., et al. “Antibodies against GD2 ganglioside can eradicate syngeneic cancer micrometastases”, Cancer Res., 1998, 58(13):2844-9.) On the other hand, randomized trials with a GM2-KLH vaccine that consistently induces IgM and IgG antibodies against GM2 in melanoma patients have demonstrated either no benefit or an initial decrease in overall survival compared with no treatment controls. (Kirkwood J. M., et al. “High-dose interferon alfa-2b significantly prolongs relapse-free and overall survival compared with the GM2-KLH/QS-21 vaccine in patients with resected stage IIB-III melanoma: results of intergroup trial E1694/S9512/C509801”, J. Clin. Oncol., 2001, 19(9):2370-80; Tarhini A. A., et al., “Prognostic significance of serum S100B protein in high-risk surgically resected melanoma patients participating in Intergroup Trial ECOG 1694”, J. Clin. Oncol., 2009, 27(1):38-44; Eggermont A. “EORTC 18961: Post-operative adjuvant ganglioside GM2-KLH21 vaccination treatment vs observation in stage II (T3-T4N0M0) melanoma: 2nd interim analysis led to an early disclosure of the results”, J. Clin. Oncol., 2008, May 20 suppl; abstr 9004; Eggermont A., et al. “Randomized Phase III Trial comparing Post-Operative Adjuvant Ganglioside GM2-KLH-QS 21 Vaccination Treatment vs Observation in Stage II (T3-T4N0M0) Melanoma: Final results of the EORTC 18961 study”, J. Clin. Oncol., 2010, 28:7, abstr 8505). GM2 is present in essentially all melanomas, but unlike GD3 and GM3, which are the most highly expressed melanoma gangliosides, it is expressed at only low levels in the majority of cases, and very few melanoma cell lines can be lysed with mAbs or immune sera against GM2 and complement. (Hamilton W. B., et al. “Ganglioside expression on human malignant melanoma assessed by quantitative immune thin-layer chromatography”, Int. J. Cancer, 1993, 53(4):566-73; Tsuchida T., “Gangliosides of human melanoma”, Cancer, 1989, 63(6):1166-74; Zhang S., et al, “Increased tumor cell reactivity and complement-dependent cytotoxicity with mixtures of monoclonal antibodies against different gangliosides”, Cancer Immunol. Immunother., 1995, 40(2):88-94.)

It has been surprisingly discovered that while high doses (i.e., sufficient titer) of many mAb anti-cancer treatments can effectively trigger complement-mediated (i.e., CDC and ADCC) cancer cell cytotoxicity, low doses or levels of the same antibodies either have no effect or result in acceleration of cell division and tumor growth. Likewise, as a therapeutically effective high dose of mAb is metabolized and its levels decrease, the resulting low levels may perpetuate survival and proliferation of remaining cancer cells, thereby diminishing net therapeutic efficacy. Moreover, mAbs directed against antigens with only low levels of cell surface expression are effectively “low dose” treatments regardless of the dose actually administered because antigen expression serves as a limiting factor for therapeutic efficacy. In other words, the tumor cell antigen density may be too low to enable formation and attachment of proteins required for complement activation. Likewise, cancer vaccines may be rendered ineffective if the antigen in the vaccine is not sufficiently expressed by the targeted cancer and/or the vaccine fails to induce sufficient titer to trigger lytic complement activation.

The present invention discloses, however, that sublytic complement activation resulting from low levels of complement-activating mAb and/or administration of a mAb against a tumor cell antigen with low density, surprisingly, activates internal cell survival pathways. This results in PI3K-mediated inflammation, angiogenesis, and tumor cell activation. It has been further discovered that the negative effects of low mAb dose levels, whether caused by metabolism of a once therapeutically effective dose or administration of a mAb against an antigen with low tumor cell density or the action of membrane-bound complement regulatory proteins (mCRP), are mediated through the PI3K/AKT pathway and can be ameliorated by administration of at least one PI3K or AKT inhibitor. Inhibition of the PI3K/AKT pathway also improves the complement-mediated high dose (i.e., lytic complement-activating) mAb treatment, significantly increasing therapeutic efficacy. Thus, concurrent administration of a PI3K or AKT inhibitor with a passively administered, complement-activating, anti-tumor mAb potentiates therapeutic efficacy. In embodiments of invention, any complement-activating, anti-tumor antibody may be concurrently administered with a specific or non-specific PI3K inhibitor.

It has been further discovered, in accordance with the present invention, that this paradigm applies to monovalent and polyvalent anti-cancer vaccines. Concurrent administration of a specific or non-specific PI3K inhibitor with a cancer vaccine capable of inducing complement-activating antibodies against a cancer antigen potentiates the therapeutic efficacy of the antibodies induced by the vaccine. Thus, if used in a setting of high-antigen-expressing tumors, monovalent vaccines should be beneficial, not detrimental, and polyvalent vaccines inducing antibody titer against several cell surface antigens should be even more beneficial.

One embodiment of the present invention involves methods of potentiating antibody-based cancer treatments. The methods comprise administering to a subject a therapeutically effective amount of a complement-activating antibody against a cancer antigen and concurrently administering a PI3K inhibitor to the subject. The invention further provides a method of treating cancer and inhibiting tumor growth. These embodiments involve the administration of a therapeutically effective amount of an anti-tumor mAb and at least one specific or non-specific PI3K inhibitor to a subject (including, but not limited to a human or animal) in need thereof.

In some embodiments of the invention, anti-tumor, complement-activating antibodies are directed against cancer antigens. Cancer antigens are expressed exclusively, significantly or abnormally on cancer cells and/or tumors relative to normal tissues. An antigen may be a protein, polypeptide, protein or polypeptide fragment, peptide, dominant epitope peptide that binds to an HLA class I or II molecule, a monosaccharide, a polysaccharide or nucleic acid. In some embodiments of the invention, these antigens are gangliosides; i.e. molecules composed of a glycosphingolipid (ceramide and oligosaccharide) with one or more sialic acids (e.g. n-acetylneuraminic acid, NANA) linked on the sugar chain. For example, monoclonal antibodies against GM2, GD2, GD3 and fucosyl GM1 may be passively administered or vaccine-induced. These antigens are generally targets in melanoma, neuroblastoma, and sarcoma. In some embodiments, the tumor-specific antigen is CD20. Though expressed at many stages of B cell development, CD20 is not expressed on plasma cells. CD20 is, however, highly expressed on B-cell lymphomas, hairy cell leukemia, B-cell chronic lymphocytic leukemia, and melanoma cancer stem cells. In some embodiments, the antigen is N-Glycolylneuraminic acid (Neu5Gc). Low doses of naturally present, affinity-purified human anti-Neu5Gc antibodies accelerate growth of Neu5Gc-containing tumors in Neu5Gc-deficient mice (Hedlund M., et al., “Evidence for a human-specific mechanism for diet and antibody-mediated inflammation in carcinoma progression”, Proc. Natl. Acad. Sci. USA, 2008, 105(48):18936-41), while at higher doses these same antibodies elicited tumor cytotoxicity (Padler-Karavani V., et al., “Human xeno-autoantibodies against a non-human sialic acid serve as novel serum biomarkers and immunotherapeutics in cancer”, Cancer Res., 2011, 71(9):3352-63). Additional cancer antigens against which antibodies of the invention may be directed or induced include Lewis Y (breast, ovary, prostate and small cell lung cancers), sialyl Lewis A (gastrointestinal malignancies), Globo H (breast, ovary and small cell lung cancer), TF (breast, ovary and prostate), Tn (breast and prostate), sialylated Tn, MUC1 (breast and ovary), KSA (breast, ovary, prostate and small cell lung cancers), and polysialic acid (small cell lung cancer and neuroblastoma). Yet more additional cancer antigens against which antibodies of the invention may be directed or induced include Erb B2 (breast), CD52 (chronic lymphocytic leukemia), epidermal growth factor receptor (EGFR, colorectal cancer), MART-1 (melanoma), gp100 (melanoma), HER2/neu (breast and epithelial cancers); carcinoembryonic antigen (CEA; bowel, lung and breast cancers), CA-125 (ovarian cancer), epithelial tumor antigen (ETA; breast cancer); NY-ESO-1 (testes and various tumors), PSA or PSMA (prostate cancer), thymus-leukemia antigen (TL), and proteins of the melanoma-associated antigen family (MAGE; hepatocellular cancer and other tumors); and components involved in angiogenesis, such as vascular endothelia growth factor (VEGF, expressed in angiogenic stroma and tumor cells), VEGF receptor 2, Id2, Id3, and Tie-2 (preferentially expressed during neoangiogenesis and in colorectal cancers). Further cancer-associated antigens may be selected, in accordance with the guidance provided herein, by those of skill in the art. General reviews for cancer antigens useful as either mAb or cancer vaccine targets of the invention include Cheever, M. A. et al., “The Prioritization of Cancer Antigens: A National Cancer Institute Pilot Project for the Acceleration of Translational Research”, Clin. Cancer Res., 2009, 15:5323-5337; Ragupathi, G. and Livingston, P., “The case for polyvalent cancer vaccines that induce antibodies”, Expert Rev. Vaccines, 2002, 1(2):89-102.

It should be appreciated that embodiments of the invention are not limited to any particular type of cancer. Any cancer that may be targeted by complement-activating antibodies, or against which complement-activating antibodies may be induced by vaccine, can be treated by the methods disclosed herein. Stated another way, any cancer treatment comprising complement-activating antibodies (preferably monoclonal) may benefit from concurrent administration of a specific or non-specific PI3K inhibitor.

Embodiments of the present invention encompass any complement-activating anti-tumor antibody. Some embodiments of the present invention utilize anti-tumor mAbs capable of inducing complement-mediated cytotoxicity. It will be appreciated by those of skill in the art that not all antibodies are capable of inducing complement-mediated cytotoxicity. The nature of the antibody being administered determines whether complement will be activated. IgM antibodies are particularly effective because they possess multiple antigen-binding sites; i.e., two adjacent antigens can be bound by a single IgM molecule. Certain IgG subclasses are also capable of activating complement: IgG subclasses 1, 2, and 3. Antibodies of both human and mouse origin, as well as chimeric antibodies, may be used in embodiments of invention. In general, the following isotypes efficiently fix human complement: mouse IgG2a, mouse IgG2b, mouse IgG3, mouse IgM, human IgG1, human IgG4 and human IgM. Effective complement-activating antibodies may be generated, induced or directed against the cancer antigens disclosed herein (e.g. glycolipids such as GM2, GD2, GD3, fucosyl GM1, globo H, and Lewis Y). In some embodiments of the invention, anti-tumor antibodies are passively administered. In some embodiments, the anti-tumor antibodies are 3F8, 5B1, R24 and PGNX.

Since FDA approval of monoclonal antibodies such as rituximab (Rituxan®) and trastuzumab (Herceptin®), and their widespread use, there is clinical value in maximizing immune effector mechanisms such as complement activation and ADCC, which these antibodies mediate. (See Zhang H. et al. “Antibodies against GD2 ganglioside can eradicate syngeneic cancer micrometastases”, Cancer Res., 1998, 58(13):2844-9; Zhou X., et al., “The role of complement in the mechanism of action of rituximab for B-cell lymphoma: implications for therapy”, Oncologist, 2008, 13(9):954-66.). In particular embodiments of the inventions, the anti-tumor, complement-activating antibodies are rituximab and trastuzumab. Additional anti-tumor antibodies utilized in the present invention include alemtuzumab (Campath), bevacizumab (Avastin®, Genentech); cetuximab (Erbitux®, Imclone); panitumumab (Vectivix®, Amgen), pertuzumab (Omnitarg®, Genentech), tositumomab (Bexxar, Corixia), and the antibody drug conjugate, gemtuzumab ozogamicin (Mylotarg®, Wyeth). Anti-tumor antibodies may also include Zamly™, epratuzumab, Cotara™, edrecolomab, bevacizumab, mitomomab, tositumomab (Bexxar®) CeaVac™ ibritumomab (Zevalin™) and OvaRex (Zevalin®). (See also, Galluzzi, L. et al., “Monoclonal antibodies in cancer therapy”, OncoImmunology, 2012, 1:28-37.)

Embodiments of the present invention and methods disclosed herein can include any antibody now known or later discovered that binds to a cancer antigen and is capable of activating complement. These antibodies may be naturally occurring, vaccine-induced, or generated by methods well known in the art. Various hosts, including goats, rabbits, rats, mice etc., may be immunized by injection of a cancer antigen. Adjuvants (e.g., Freund's) may be used to increase the immunological response. To generate polyclonal antibodies, the cancer antigen(s) may be conjugated to a conventional carrier to increase immunogenicity, and anti-serum to the antigen raised. Techniques for preparing monoclonal antibodies are well known in the art (see, e.g., Arnheiter et al., 1981, Nature, 294:278). Monoclonal antibodies may be obtained from hybridoma tissue cultures or from ascites fluid obtained from animals into which the hybridoma tissue was introduced.

Antibodies within the scope of the invention, particularly human antibodies, can be derived from antibody libraries. Many of the difficulties associated with generating monoclonal antibodies by B-cell immortalization can be overcome by engineering and expressing antibody fragments in E. coli, using phage display. To ensure the recovery of high affinity monoclonal antibodies, a combinatorial immunoglobulin library must typically contain a large repertoire size. A typical strategy utilizes mRNA obtained from lymphocytes or spleen cells of immunized mice to synthesize cDNA using reverse transcriptase. The heavy- and light-chain genes are amplified separately by PCR and ligated into phage cloning vectors. Two different libraries are produced, one containing the heavy-chain genes and one containing the light-chain genes. Phage DNA is isolated from each library, and the heavy- and light-chain sequences are ligated together and packaged to form a combinatorial library. Each phage contains a random pair of heavy- and light-chain cDNAs and upon infection of E. coli directs the expression of the antibody chains in infected cells. To identify an antibody that recognizes the antigen of interest, the phage library is plated, and the antibody molecules present in the plaques are transferred to filters. The filters are incubated with radioactively labeled antigen and then washed to remove excess unbound ligand. A radioactive spot on the autoradiogram identifies a plaque that contains an antibody that binds the antigen. Antibodies for use in some embodiments of the invention may be derived from yeast display libraries (see, e.g., International Publication WO2009/036379).

In general, humanized or veneered antibodies minimize unwanted immunological responses that limit the duration and effectiveness of therapeutic applications of non-human antibodies in human recipients. A number of methods for preparing humanized antibodies comprising an antigen binding portion derived from a non-human antibody have been described in the art. In particular, antibodies with rodent variable regions and their associated complementarity-determining regions (CDRs) fused to human constant domains have been described (see, e.g., Winter et al., Nature 349:293, 1991; Lobuglio et al., Proc. Nat. Acad. Sci. USA 86:4220, 1989; Shaw et al., J. Immunol. 138:4534, 1987; and Brown et al., Cancer Res. 47:3577, 1987). Rodent CDRs grafted into a human supporting framework region (FR) prior to fusion with an appropriate human antibody constant domain (e.g., see Riechmann et al., Nature 332:323, 1988; Verhoeyen et al., Science 239:1534, 1988; and Jones et al. Nature 321:522, 1986) and rodent CDRs supported by recombinantly veneered rodent FRs have also been described (e.g., see EPO Patent Pub. No. 519, 596). Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes (e.g., see Lonberg and Huszar Int. Rev. Immunol. 13:65-93, 1995 and U.S. Pat. Nos. 5,545,806; 5,569,825; 5,625,126; 5,633,425; and 5,661,016). Veneered versions of the provided antibodies may also be used in the methods of the present invention. The process of veneering involves selectively replacing FR residues from, e.g., a murine heavy or light chain variable region, with human FR residues in order to provide an antibody that comprises an antigen binding portion which retains substantially all of the native FR protein folding structure. Veneering techniques are based on the understanding that the antigen binding characteristics of an antigen binding portion are determined primarily by the structure and relative disposition of the heavy and light chain CDR sets within the antigen-association surface (e.g., see Davies et al., Ann. Rev. Biochem. 59:439, 1990). Thus, antigen association specificity can be preserved in a humanized antibody only wherein the CDR structures, their interaction with each other and their interaction with the rest of the variable region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

Embodiments of the invention may involve administration of mAbs by means and dosages known to those of skill in the art. Various routes of administration may be employed for dosing mAbs used in embodiments of the invention. Routes of mAb administration may be, for example, intravenous, subcutaneous, intramuscular, oral, or via inhalation.

Those of skill in the art will appreciate that a characteristic portion of an mAb may, in some embodiments, be sufficient to implement complement-mediated cytoxicity. In certain embodiments, an antibody fragment may be used that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively or additionally, an antibody fragment may comprise multiple chains which are linked together, for example, by disulfide linkages. Select antibodies and antibody fragments may be used individually or in combination. When used in combination, the select antibodies and antibody fragments may be used simultaneously or sequentially.

In some embodiments of the invention, a high dose of anti-tumor mAb is concurrently administered along with a PI3K inhibitor to increase the effectiveness of or potentiate the mAb treatment. In some embodiments, a high dose is between about 1-150 milligrams of anti-tumor antibody per kilogram (kg) of body weight of the subject. In some embodiments, a high dose is between about 15-150 milligrams of anti-tumor antibody per kilogram (kg) of body weight of the subject. In particular embodiments, a high dose of mAb is about 40-50 milligrams per kilogram of body weight of a subject when a mAb directed against GM2, GD2, GD3, CD20, sialyl Lewis A (“sLe^{a}”) or Neu5Gc is administered to the subject. Methods and dosages of mAb-based cancer treatments have been described previously. (See, e.g., Adams, G. P. and Weiner, L. M., “Monoclonal Antibody Therapy of Cancer”, Nature Biotech., 2005, 23:1147-57; Oldham, R. K., et al., “Monoclonal Antibodies in Cancer Therapy: 25 Years of Progress”, J. Clinical Oncol., 2008, 26(11):1774-1777, and articles cited therein)

In some embodiments of the invention, the anti-tumor antibodies are induced against a cancer antigen by a cancer vaccine. All vaccines that induce complement-dependent tumor cell death are encompassed within embodiments of the invention. In general, cancer vaccines according to embodiment of the invention may be designed to induce antibodies against any of the aforementioned cancer antigens. In particular embodiments, cancer vaccines according to embodiments of the invention may comprise one or more antigens selected from the group consisting of GM2, GD2, GD3 and fucosyl GM1; glycolipids such as Lewis Y, sialyl Lewis A and Globo H; mono- or disaccharide antigens O-linked to mucins such as Thomsen-Friedenreich antigen (“TF”), Tn and sialylated Tn; Mucin 1 (“MUC1”); adenocarcinoma-associated antigen (“KSA”); prostate-specific antigen (“PSMA”); polysialic acid, and CA125. Cancer vaccines may also unimolecular, multiantigenic constructs, including STn cluster, TN cluster and TF clustered antigens (see, e.g., Zhu, J., et al., Expert Rev Vaccines, 8: 1399-1413, 2009; Ragupathi, G. et al., J. Am Chem Soc., 128: 2715-2725, 2006, incorporated by reference herein). Cancer vaccines and methods of producing cancer vaccines are known in the art. (See, e.g., Ragupathi, G. and Livingston, P., “The case for polyvalent cancer vaccines”, Expert Rev. Vaccines, 2002, 1(2):89-102; Kim, S. K. et al. “Effect of immunological adjuvant combinations on the antibody and T-cell response to vaccination with MUC1-KLH and GD3-KLH conjugates”, Vaccine, 2001, 19:530-537; “Comparison of the effect of different immunological adjuvants on the antibody and T-cell response to immunization with MUC1-KLH and GD3-KLH conjugate cancer vaccines”, Vaccine, 2000, 18:597-603; Helling, F. et al., “GD3 Vaccines for Melanoma: Superior Immunogenicity of Keyhole Limpet Hemocyanin Conjugate Vaccines”, Cancer Res., 1994, 54:197-203).

The effectiveness of a cancer vaccine may be directly related to the vaccine's ability to generate antibodies capable of causing CDC and/or ADCC. Concurrent administration or a pre-/post-vaccination dosing regimen of a PI3K inhibitor may potentiate complement-mediated cell death, thus allowing lower antibody titers to be effective. Additionally or alternatively, administration of a PI3K inhibitor may allow a lower dose of antigen to be administered. As certain antigens may be auto-antigens expressed to some degree on a variety of normal tissues, it may be desirable to administer as low an antigen dose as possible to avoid provoking an auto-immune response. Additionally, embodiments of the invention may potentiate the effectiveness of cancer vaccines that include antigens that are marginally expressed in a given cancer.

Cancer vaccines according to embodiments of the invention may be monovalent or polyvalent. Polyvalent vaccines may be required due to tumor cell heterogeneity, heterogeneity of the human immune response, and the correlation between overall antibody titer against tumor cells and antibody effector mechanisms. A pre-vaccination, concurrent administration or post-vaccination dosing regimen of at least one PI3K inhibitor may potentiate antibody effector mechanisms, thereby increasing the effectiveness of both polyvalent and monovalent vaccines. Polyvalent vaccines may comprise also unimolecular, multiantigenic constructs, as described above.

The induction of active immunity against certain cancer antigens can be more difficult than induction of immunity against viral or bacterial antigens because tumor antigens may be expressed to some degree, or in slightly modified form, in normal tissues. Thus, in some embodiments of the invention, cancer vaccines comprise covalent attachment of a cancer antigen to an immunogenic carrier molecule. In certain embodiments, the carrier molecule may be selected from the group consisting of Keyhole Limpet Hemocyanin (“KLH”), Neisseria meningitidis outer membrane proteins, multiple antigenic peptide, cationized bovine serum albumin and polylysine.

Cancer vaccines according to embodiments of the invention may also comprise one or more adjuvants Immunologic adjuvants for use in embodiments of the invention include CRL-1005 (polypropylene), CpG ODN 1826 (synthetic bacterial nucleotide), GM-CSF (peptide), MPL-SE (monophosphoryl lipid A), GPI-0100 (hydrolyzed saponin fractions), MoGM-CSF (F_{c}-GM-CSF fusion protein), PG-026 (Peptidoglycan), QS-21 (saponin fraction), synthetic QS-21 analogs, and TiterMax Gold (CRL-8300 (polyoxypropylene; polyoxyethylene).

In some embodiments of the invention, a PI3K inhibitor is concurrently administered with an anti-tumor antibody or cancer vaccine to potentiate the therapy and/or overcome an increase in cell survival or proliferation caused by the “low dose” effect. As discussed above, this effect can occur because of: (1) low expression of the antigen against which the mAb is directed; and (2) metabolism of a therapeutically effective dose that diminishes levels of the mAb below that necessary for complement activation. In some embodiments, a “low dose” effect may be observed when there is little or no detectable serum antibody within 2-4 hours of dosing. In some embodiments, a “low dose” effect is correlated with antibody levels between about 0.01-1.0 milligrams of anti-tumor antibody per kilogram (kg) of body weight of the subject. In some embodiments, a low dose is correlated with antibody levels between about 0.001-1.0 milligrams of anti-tumor antibody per kilogram (kg) of body weight of the subject.

In some embodiments of the invention, multiple anti-tumor antibodies may be co-administered or concurrently administered as a combination therapy. Concurrent administration may involve separate but simultaneous administration of two or more anti-tumor mAbs. In other embodiments, concurrent administration involves sequential administration wherein administration of one mAb immediately or approximately precedes administration of another mAb. In some embodiments, one or more mAbs may be administered as part of a dosing regimen involving repeated administration of the same one or more mAbs. Concurrent administration may also entail combined administration as a single unit dose.

Some embodiments of the invention comprise administration of an anti-tumor mAb as part of an overall cancer treatment regimen in which cytotoxic or chemotherapeutic agents are also administered. In some embodiments, an anti-tumor mAb and PI3K inhibitor are concurrently administered with a cytotoxic or chemotherapeutic agent. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, camomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Embodiments of the present invention encompass a variety of modes of administration and dosages of the therapeutic agents disclosed herein. Both mode of administration and dosage may vary with the particular stage of the cancer being treated, the age and physical condition of the subject being treated, the duration of the treatment, the nature of any concurrent therapy, the specific route of administration, and the like. Appreciation of these factors and their effects are well within the knowledge and expertise of health practitioners.

Embodiments of the invention require specific or non-specific inhibition of the PI3K pathway. Phosphoinositide 3-kinases (PI3K) are lipid kinases that phosphorylate lipids at the 3-hydroxyl residue of an inositol ring (Whitman et al (1988) Nature, 332:664). There are three classes of PI3K, each with its own substrate specificity and distinct lipid products. The Class IA of PI3Ks is widely implicated in cancer. PI3K activation initiates a signal transduction cascade that promotes cancer cell growth, survival and metabolism. PI3K themselves are composed of regulatory subunits (p85) and catalytic subunits (p110). There are five variants of the p85 regulatory subunit, designated p85α, p55α, p50α, p85β, or p55γ. There are also three variants of the p110 catalytic subunit designated p110α, β, or δ catalytic subunit. The most highly expressed regulatory subunit is p85α. In regard to the catalytic subunit, the first two p110 isoforms (α and β) are expressed in all cells, but p110δ is expressed primarily in leukocytes.

The 3-phosphorylated phospholipids (PIP3s) generated by PI3-kinases act as second messengers recruiting kinases with lipid binding domains (including plekstrin homology (PH) regions), such as Akt (a serine-threonine kinases) and phosphoinositide-dependent kinase-1 (PDK1). There are three different isoforms of Akt (Akt1-3) that have both overlapping and distinct roles in cancers. Binding of Akt to membrane PIP3s causes the translocation of Akt to the plasma membrane, bringing Akt into contact with PDK1, which is responsible for activating Akt. Akt1 is involved in cellular survival pathways and can inhibit apoptosis. Although Akt is the PI3K effector most widely implicated in cancer, there are Akt-independent pathways activated by PI3K. These include the Bruton tyrosine kinase (BTK); the Tec families of non-receptor tyrosine kinases; serum- and glucocorticoid-regulated kinases (SGKs); and regulators of small GTPases that are implicated in cell polarity and migration. In some embodiments of the invention, a PI3K inhibitor may act against these Akt-independent pathways.

At the molecular level, receptor tyrosine kinase (RTK) signaling often activates PI3Ks, although the p110β-containing enzymes might also be activated by G protein-coupled receptors. The p85 regulatory subunit is crucial in mediating class I PI3K activation by RTKs. The Src-homology 2 (SH2) domains of p85 bind to phosphotyrosine residues in the sequence context pYxxM (in which a ‘pY’ indicates a phosphorylated tyrosine) on activated RTKs. This binding of SH2 domains serves both to recruit the p85-p110 heterodimer to the plasma membrane, where its substrate PIP2 resides, and to relieve basal inhibition of p110 by p85. The 3′-phosphatase PTEN dephosphorylates PIP3 and therefore terminates PI3K signaling.

Accumulation of PIP3 on the cell membrane leads to the colocalization of signaling proteins with pleckstrin homology (PH) domains. This leads to the activation of these proteins and propagation of downstream PI3K signaling. Akt and phosphoinositide-dependent protein kinase 1 (PDK1) directly bind to PIP3 and are thereby recruited to the plasma membrane. The phosphorylation of Akt at T308 (which is in the activation loop of Akt) by PDK1 and at 5473 (which is in a hydrophobic motif of Aid) by mTOR complex 2 (mTORC2) results in full activation of this protein kinase. In turn, Akt phosphorylates several cellular proteins, including glycogen synthase kinase 3α (GSK3α), GSK3β, forkhead box O transcription factors (FoxO), MDM2, BCL2-interacting mediator of cell death (BIM) and BCL2-associated agonist of cell death (BAD) to facilitate cell survival and cell cycle entry. In addition, Akt phosphorylates and inactivates tuberous sclerosis 2 (TSC2), a GTPase-activating protein for Ras homologue enriched in brain (RHEB). Inactivation of TSC2 allows RHEB to accumulate in the GTP-bound state and thereby activate mTORC1. The PI3K pathway through Akt also regulates the use and uptake of glucose. The mTOR complex 1 (mTORC1) is a major effector of Akt signaling. Not only is it activated by PI3K-Akt signaling, mTORC1 also integrates many inputs, including growth factor signaling, AMP levels and nutrient and O₂ availability.

In some embodiments of the invention, one or more PI3K inhibitors may be administered through a variety of dosing regimens. PI3K inhibitors for use in embodiments of the invention may inhibit activation of or interfere with the catalytic activity of any component of the PI3K pathway. For example, inhibitors for use in embodiments of the invention may inhibit the p110 catalytic subunit or Akt. In some embodiments of the invention, a PI3K inhibitor may block a downstream effector, such as MDM2. A PI3K inhibitor may also increase the activity or expression of PTEN, which terminates PI3K signaling. In some embodiments, a PI3K inhibitor may directly affect both PI3K and mTOR, whereas others inhibit only PI3K or only mTOR. In some embodiments, a PI3K inhibitor interferes with the PI3K pathway and one or more additional signal transduction pathways. In some embodiments, the mTOR inhibitor rapamycin is used. In some embodiments, a PI3K inhibitor is specific for all of the catalytic or regulatory subunit isoforms of class IA PI3Ks; e.g. p110α, p110β and p110δ or p85α. In other embodiments, an inhibitor may be specific only for individual isoforms. Likewise, in embodiments where Akt is inhibited, an inhibitor may block or interfere with all the isoforms of Akt, or an inhibitor may be specific for a given variant. Specific examples of PI3K inhibitors include Wortmannin, LY294002, LY49002, SF-1126 (Semafore Pharmaceuticals), BEZ235 and BKM120 and BYL719 (Novartis), XL-147 (Exelixis, Inc.), GDC-0941 (Plramed and Genentech) and combinations thereof. BEZ235 is a PI3K/mTOR dual inhibitor; BKM120 is a pan-PI3K inhibitor; and BYL719 selectively inhibits PI3Kα. These compounds have shown significant cell growth inhibition and induction of apoptosis in a variety of tumor cell lines as well as in animal models. (Maira S. M., et. al. “Identification and development of BEZ235, a new orally available dual PI3K/mTOR inhibitor with potent in vivo antitumor activity”, Mol. Cancer Ther., 2008, 7:1851-1863; Serra V., et al. “BEZ235, a dual PI3K/mTOR inhibitor, prevents PI3K signaling and inhibits the growth of cancer cells with activating PI3K mutations”, Cancer Res., 2008, 68:8022-8030; Engelman J. A., et al. “Effective use of PI3K and MEK inhibitors to treat mutant Kras G12D and PIK3CA H1047R murine lung cancer”, Nat. Med., 2008, 14:1315-1316.) Other PI3K/Akt inhibitors for use in embodiments of the invention include BGT226 (Novartis), GSK1059615 and GSK690693 (GSK), XL-765 (Exelis), PX866 (Oncothyreon), GDC0941 (Genentech/Piramed/Roche), CAL101 (Calistoga Pharmaceuticals), Perifosine (Keryx), VQD002 (Vioquest), BAY80-6946 (Bayer), PF-05212384 (Pfizer) and MK2206 (Merck). In some embodiments, multiple PI3K inhibitors may be concurrently administered either separately or in combination, before, during and/or after administration of an anti-tumor antibody.

In general, an effective amount of a PI3K inhibitor is any amount that alone, or in combination with further doses of the same or different inhibitor, inhibits or slows cell growth and/or promotes complement-mediated cytotoxicity (i.e., CDS or ADCC) of cancerous cells. In some embodiments, dosing regimens of PI3K inhibitors range include oral or parenteral administration at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In some embodiments of the invention, PI3K inhibition is achieved by interference with transcription and/or translation of genes encoding components of the PI3K pathway. For example, some embodiments of the invention utilize an interfering RNA molecule that can inhibit or down-regulate gene expression or silence a gene in a sequence-specific manner, for example by mediating RNA interference (RNAi). RNAi is an evolutionarily conserved, sequence-specific mechanism triggered by double-stranded RNA (dsRNA) that induces degradation of complementary target single-stranded mRNA and “silencing” of the corresponding translated sequences (McManus and Sharp, 2002, Nature Rev. Genet., 2002, 3: 737). RNAi functions by enzymatic cleavage of longer dsRNA strands into biologically active “short-interfering RNA” (siRNA) sequences of about 21-23 nucleotides in length (Elbashir et al., Genes Dev., 2001, 15: 188). An interfering RNA suitable for use in the practice of the present invention can be provided in any of several forms. For example, an interfering RNA can be provided as one or more of an isolated short interfering RNA (siRNA), double-stranded RNA (dsRNA), micro-RNA (miRNA), or short hairpin RNA (shRNA). RNA molecules capable of interfering with the PI3K pathway are known in the art (see, e.g., U.S. Pat. Publication No. 2005/0272682).

As with administration of the anti-tumor mAbs, dosages and dosage regimes of PI3K inhibitors may depend on the particular cancer being treated, the stage or severity of the cancer, the individual patient parameters (e.g. age, physical condition, sex, size and weight), the duration of the treatment, the nature of any concurrent therapy, and the specific route of administration. In some embodiments, multiple PI3K inhibitors may be concurrently administered. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. In some embodiments, multiple doses per day are administered to achieve appropriate systemic levels of compounds. In some embodiments, a maximum dose may be the highest safe dose according to those of skill in the art. In some embodiments, the minimum dose is the lowest dose that may be administered to overcome or inhibit the increase in cancer cell proliferation caused by low dose mAb treatment; i.e., the minimum dose may be the lowest dose that is required to allow complement-mediated cytotoxicity of low dose mAb treatments.

As described above, some embodiments of the invention encompass the concurrent administration of an anti-tumor mAb or vaccine and PI3K inhibitor as a unit dose. In some embodiments, a unit dose may be in liquid form. Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such as Cremophor, alcohols, oils, modified oils, glycols, polysorbates, cyclodextrins, polymers, and combinations thereof.

In some embodiments, a unit dose of PI3K inhibitor and anti-tumor mAb or cancer vaccine may be injected. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it may be desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which in turn may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

In some embodiments, a unit dose is in solid form. Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In some embodiments, the PI3K inhibitor(s) and/or mAbs may be administered as sustained release formulations. A sustained release formulation may comprise a biocompatible polymer, or blend of biocompatible polymers, with a PI3K inhibitor and/or mAb incorporated therein. Methods of forming sustained released compositions of active agents are known to those of skill in the art; see, e.g., U.S. Pat. No. 5,019,440 to Gombotz, et al. and U.S. Pat. No. 5,922,253 to Herbet et al, incorporated by reference herein.

It will also be appreciated that the mAbs, vaccines, PI3K inhibitors and pharmaceutical compositions of the same may be utilized in combination therapies; that is, they can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In some embodiments of the invention, a PI3K inhibitor may be concurrently administered with a complement-activating anti-tumor antibody or vaccine, and an inhibitor of another signal transduction pathway. For example, in some embodiments, an inhibitor of the MAPK/ERK kinase (“MEK”) pathway is concurrently administered. This pathway is activated by extracellular growth factors (e.g., EGF) that bind to receptors (e.g., EGF receptor) and induce a conformation change in the receptor. The conformational change leads to autophosphorylation, receptor dimerization, and recruitment of proteins such as Ras to the inner cell surface membrane. Ras stimulates Raf activation, which in turn phosphorylates MEK, when in turn activates ERK. ERK coordinates responses to the extracellular signal by regulation gene expression, cytoskeletal rearrangements, metabolism, proliferation and apoptosis. MEK inhibitors for use in embodiments of the invention may interfere with any of these activating steps or the consequences of the same. Particular MEK inhibitors for use in embodiments of the invention include AZD6244, GSK202011, PD98059, U0126, CI-1040 (PD184352) and PD0325901 (Pfizer), MEK162 and RAF265 (Novartis), ARRY-162 and ARRY-142886 (Array BioPharma), PD0325901, SL327 (Sigma-Aldrich), PD184161, sunitinib, sorafenib, Vandetanib, pazopanib, Axitinib, PTK787, PD184352, BAY 43-9006, BAY86-9766, PD325901, GSK1120212, ARRY-438162, RDEA1 19, R05126766, XL518 and AZD8330 (also ARRY-704). In some embodiments, at least one MEK inhibitor is concurrently administered with an anti-tumor complement-activating antibody or vaccine in the absence of a PI3K inhibitor. As described above for PI3K inhibition, MEK inhibitors include inhibition at the level of transcription and translation, such as by RNAi.

In addition to the treatment of cancer as described herein, some embodiments of the invention may be suitable to treat a variety of hyperproliferative, infectious or auto-immune diseases. For example, the compounds and pharmaceutical compositions of the invention may be used to treat or prevent benign neoplasms, diabetic retinopathy, rheumatoid arthritis, or lupus. Embodiments of the invention may also be used in the treatment of any disease caused, sustained or exacerbated by inactivation of the complement system.

In some embodiments of the invention, methods are provided for identifying and treating subjects suitable for cancer treatments comprising complement-activating antibodies. In general, these subjects will suffer from or be susceptible to types of cancer in which the cancerous cells express quantitatively high levels of antigens against which complement-activating antibodies may be targeted. In other words, therapies with complement-activating antibodies should be restricted to treatment of antigen-rich tumors and cells. These types of cancers may be identified by obtaining a sample from a subject and quantifying the levels of a particular antigen of interest (e.g., GM2, GD2, and GD3). The subject may be susceptible to cancer, suffer from cancer or be suspected of having cancer. The sample may be tumor cells, solid tissue, or any biological fluid in which cancer cells can be detected and isolated.

Once the sample is obtained, antigen expression can be determined by techniques known to those of skill in the art. Expression levels may be determined by both nucleic acid (e.g. mRNA) and protein measurement. For example, protein expression levels may be determined by immunoassays, Western Blot analysis, or two-dimensional gel electrophoresis. Representative immunoassays include immunohistochemistry (including tissue microarray formats), fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). Protein levels may also be detected based upon detection of protein/protein interactions, including protein/antibody interactions using techniques such as Fluorescence Correlation Spectroscopy, Surface-Enhanced Laser Desorption/Ionization Time-Of-flight Spectroscopy, and BIACORE technology. RNA expression levels may be determined using techniques such as reverse-transcriptase polymerase chain reaction (RT-PCR), quantitative reverse-transcriptase polymerase chain reaction (QRT-PCR), real-time-PCR, serial analysis of gene expression (SAGE) microarray hybridization, Northern Blot analysis, and in situ hybridization. Methods of quantifying antigen expression in tumor cells are known in the art. (See, e.g., U.S. Pat. No. 7,776,612; U.S. Pre-grant Publication No. 2009/00812125.)

The quantification of antigens may be used to determine whether the cancer cells or tumor express an antigen beyond a threshold of therapeutic efficacy. For example, whether antigen expression is sufficient may be determined by qualitatively comparing expression levels against those in normal cells or by comparing expression to levels known to activate complement. In general, the threshold of therapeutic efficacy is the point where sufficient membrane attack complexes have formed to cause cell lysis. Below this threshold, i.e., a sublytic number, cancer cells activate cell survival pathways and proliferate. Various factors affect complex formation, including antigen expression level, amount of antibody used, and expression of complement regulatory proteins (mCRP). (see, e.g., van Meerten, T. et al., “Complement-induced cell death by rituximab depends on CD20 expression level and acts complementary to antibody-dependent cellular cytotoxicity”, Cancer Res., 2006, 12(13):4027-35.) In some embodiments, expression of a given antigen at greater than 1000 copies per cell may be sufficient for complement activation. In other embodiments, expression of a given antigen at greater than 500 copies per cell may be sufficient for complement activation. In other embodiments, expression of a given antigen at greater than 250 copies per cell may be sufficient for complement activation. In some embodiments, expression of a given antigen at greater than 100 copies per cell may be sufficient for complement activation.

Unless otherwise stated, the invention makes use of standard methods of molecular biology, cell culture, animal maintenance, cancer diagnosis and treatment, and administration of therapeutic agents to subjects, etc. This application refers to various patents and publications. The contents of all scientific articles, books, patents, and other publications, mentioned in this application are incorporated herein by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of February 2012; Sambrook, Russell, and Sambrook, Molecular Cloning: A Laboratory Manual, 3^{rd} ed., Cold Spring Harbor Laboratory Press, Cold Spring Harbor, 2001; Kuby Immunology, 6^{th} ed., Goldsby, R. A., Kindt, T. J., and Osborne, B. (eds.), W.H. Freeman, 2000; Goodman and Gilman's The Pharmacological Basis of Therapeutics, 12^{th} Ed. McGraw Hill, 2010; Katzung, B. (ed.) Basic and Clinical Pharmacology, McGraw-Hill/Appleton & Lange; 9th edition (June 2010). In the event of a conflict or inconsistency between any of the incorporated references and the instant specification, the specification shall control, it being understood that the determination of whether a conflict or inconsistency exists is within the discretion of the inventors and can be made at any time.

The invention will be more fully understood by reference to the following examples. They should not, however, be construed as limiting the scope of the invention. All literature citations are incorporated by reference.

### EXAMPLES

### Example 1

### Materials and Methods

The materials and methods used in the following examples are described herein.

### Monoclonal Antibodies(mAb) and Reagents

The following anti-tumor monoclonal antibodies were used: mAb PGNX (anti-GM2, murine IgM; Progenics); mAb 3F8 (anti-GD2, murine IgG3; Memorial Sloan-Kettering Cancer Center (“MSKCC”)); mAb R24 (anti-GD3, murine IgG3; MSKCC); Rituxan (anti-CD20, chimeric IgG; Genentech); mAb 5B1. mAb against p-Aid, Aid, p-PRAS40 and PRAS40 were obtained from Cell Signaling Technology (Danvers, Mass.). PI3K inhibitors BEZ235, Wortmannin were from Chemdea (Ridgewood, N.J.). MEK inhibitor GSK1120212, AZD6422, PI3K inhibitor BKM120 and AKT inhibitor MK2206 were purchased from Selleckchem (Houston, Tex.).

### Cell Culture

CHLA136Luc, luciferase transduced CHLA136 human neuroblastoma cell line was maintained in Iscove's Modified Dulbecco's Medium supplemented with 15% FBS and ITS premix (BD Bioscience, Bedford, Mass.) at 37° C., 5% CO2 in a humidified chamber. Lan-1 neuroblastoma, Hs445 lymphoma and the small cell lung cancer cell line H524 were maintained in RPMI-1640 media supplemented with 10% FBS at 37° C. 5% CO2 in a humidified chamber. Colo205 colorectal adenocarcinoma cells were cultured under similar conditions.

### In Vivo

Animals. CB17 SCID mice (Taconic) 5-8 weeks old were housed 5 to a cage. The Memorial Sloan Kettering Cancer Center Institutional Animal Care and Use Committee (IACUC) approved all protocols and procedures.

Mouse data can be extrapolated by those of skill in the art to provide effective dosing ranges for humans. An equivalent human dose may be calculated based on a body surface area calculation published by the FDA; see, e.g., “Guidance for Industry: Estimating the Initial Maximum Safe Starting Dose In Initial Clinical Trials For Therapeutics In Healthy Adult Volunteers”, available at hup://www.fda.gov/downloads/Drugs/GuidanceComplianceRegulatoryInformation/Guidances/ucm078932.pdf, incorporated by reference herein.

Tumor Challenge. Mice were placed under a heat lamp for 3 minutes and immobilized in a mouse restrainer; 0.5 million CHLA136Luc cells in 100 μl were injected into the tail vein using a BD insulin syringe with 28 gauge needle.

mAb administration. Mice were treated with murine mAbs 3F8, PGNX, R24, against GD2, GM2, GD3 and Rituxan against CD20. Control mice, typically 2 cages of 5 mice, were treated identically, receiving the same volume of PBS at the same intervals.

Imaging. Mice were anaesthetized using isoflurane and injected with 300 μg of D-Luciferin Firefly (Caliper LifeScience, Hopkinton, Mass.). They were imaged 10 minutes later using the IVIS200 in vivo imaging system (Caliper Life Science) over periods of time ranging up to 3 minutes using the software program “Living Image 3.0” (Caliper Life Science). Values are reported as photons/second.

### In Vitro

ELISA assay. ELISA assays were performed to determine IgM and IgG serum antibody titers against GM2, GD2, and GD3 after administration of mAbs targeting these gangliosides. Briefly 0.1 μg ganglioside per well in ethanol was coated on ELISA plates overnight at room temperature. Nonspecific sites were blocked with 3% human serum albumin in saline for 2 hours. Serially diluted sera drawn at intervals after mAb administration were added to each well. After 1 hour incubation, the plates were washed and alkaline phosphatase-labeled goat anti-mouse IgM or IgG added at 1:200 dilution. The antibody titer was defined as the highest dilution with absorbance of ≧0.1 over that of control mouse sera. Pretreatment sera were consistently negative (absorbance <0.1 at a dilution of 1/5).

FACS. Flow cytometry with the indicated cultured cancer cell lines was performed as described (Ragupathi G. et al. “Antibodies against tumor cell glycolipids and proteins, but not mucins, mediate complement-dependent cytotoxicity”, J. Immunol., 2005, 174(9):5706-12). In brief, single cell suspensions of 1×10⁶ culture tumor cells per tube were washed in PBS with 3% fetal bovine serum (FBS). Murine monoclonal antibodies PGNX (IgM against GM2), 3F8 (IgG3, GD2), R24 (IgG3, GD3), and Rituxan, (IgG1, CD20) were used to identify the respective antigens. After wash in 3% FBS, 20 μl of 1:25 diluted goat anti-mouse IgM or IgG labeled with fluorescein-isothiocyanate (FITC, Southern Biotechnology, Birmingham, Ala.) was added, and the mixture incubated for another 30 minutes on ice. After a final wash, the positive population and median fluorescence intensity of stained cells were differentiated using FACS Scan (Becton & Dickinson, San Jose, Calif.). Cells stained only with goat anti-mouse IgM or IgG labeled with fluorescein-isothiocyanate were used to set the FACScan result at 1% as background for comparison to percent positive cells stained with primary mAbs.

WST-1 assay. WST-1 cell proliferation assay kit was used for detection of the extent of cellular proliferation according to the company's manual. Briefly, 20,000 cells in 100 μl of culture media as defined above were plated in a 96 well flat bottom plate and incubated at 37° C. in 5% CO₂ overnight. Antibody doses between 0.02 ρg to 5 μg in 1 μl of defined culture media were added to each well and incubated for 1 hour at 37° C., 5% CO₂; 4-10 μl of human serum complement (Quidel Corp. San Diego, Calif.) was then added to each well and incubated overnight. For assays testing the impact of PI3K inhibitor, BEZ235 (Chemdea, Ridgewood, N.J.) at 0.005, 0.5 or 5.0 μg/ml were added accordingly at same time when mAb was added. WST-1 agent (Roche Applied Science, Indianapolis Ind.) was added at 1:10 ratio at the end of incubation, and OD (Optical density) was acquired by reading the plates at 415 nm 4 hours later. The Student t test was used for statistical analysis.

Western blot. 1×10⁶ Cells were plated into 6 well plates and incubated overnight. Cells were then treated with BEZ235, mAbs and human sera complement at the dose indicated for 4 hours. At the end of incubation, cells were collected and lysed with lysis buffer from Cell Signal (Danvers, Mass.), which contains protease inhibitor cocktail and phosphatase inhibitor cocktail (Calbiochem, Philadelphia, Pa.), each at 1:100 dilution (Cocktails:lysis buffer). The cell lysates were then quantitated using Bradford assay (Bio-Rad, Hercules, Calif.) according to that company's manual: 30 μg of cell lysate protein from each sample were running on 7.5% of Tris-HCL gel (Bio-Rad) and transferred to a PVDF membrane. Membrane was then blocked with Pierce blocking buffer overnight at 4° C., probed with indicated mAbs at 1:1000 dilution overnight at 4° C. and HRP-goat anti-rabbit-IgG antibody at 1:1000 for 1 hour. The membrane was washed with PBS-T (0.1% Tween-20+PBS) 5 minutes on a shaker 5 times after each incubating and then developed using Amersham™ ECL™ Prime Western Blotting Detection Reagent (GE Healthcare, Piscataway, N.J.). Imaging was acquired by scanning the membrane on the FujiFilm LAS-3000 Imager.

Statistical analysis. Overall survival was defined as the time from IV tumor cell challenge to date of death or day 160. Survival distributions were generated using Kaplan-Meier methodology (Kaplan, “Nonparametric estimation from incomplete observations”, J. Am. Stat. Assoc., 1958, 53:457-81) and comparisons between treatment group and control (PBS) were made via the Student t test (using Graphpad Prism 5).

### Example 2

### Confirmation of Antigen Expression on Target Cell Lines

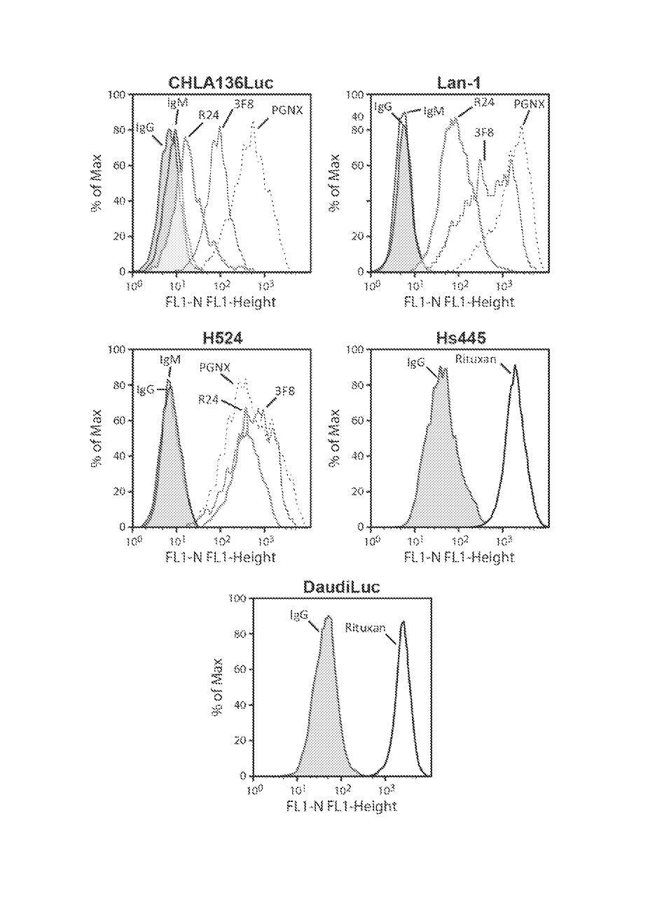

Cell surface expression of GM2, GD2 and GD3 on neuroblastoma cell lines CHLA136 and Lan-1 and SCLC cell line H524, and CD20 expression on lymphoma cell lines Hs445 and Daudi were confirmed by flow cytometry (FIG. 1).

### Example 3

### In Vivo Experiments Targeting GM2, GD2, GD3, and CD20

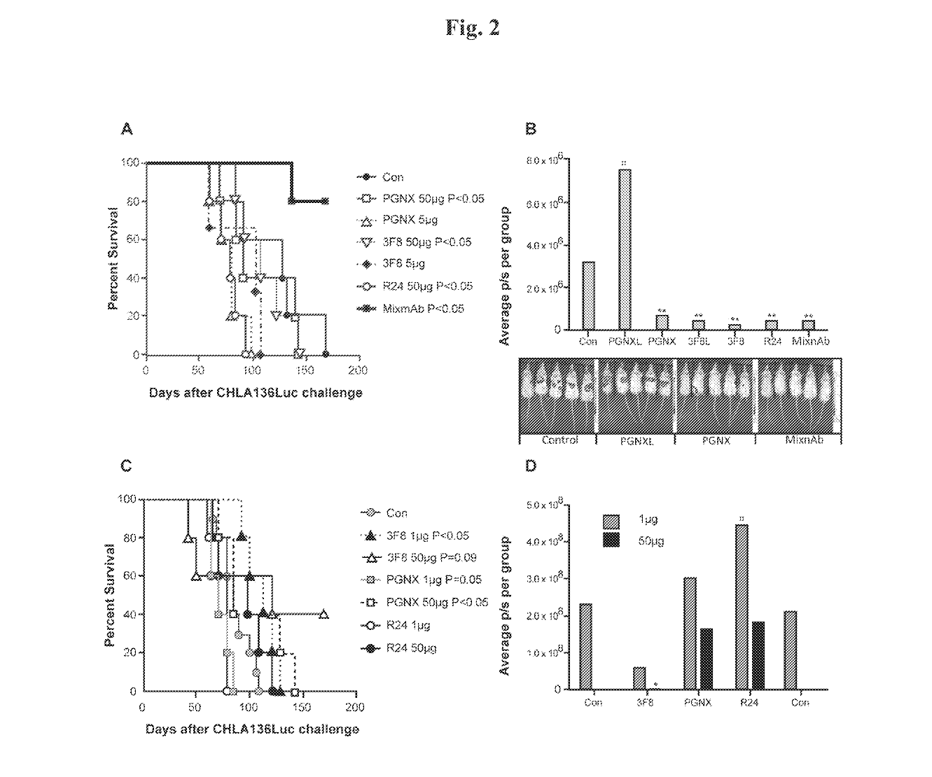

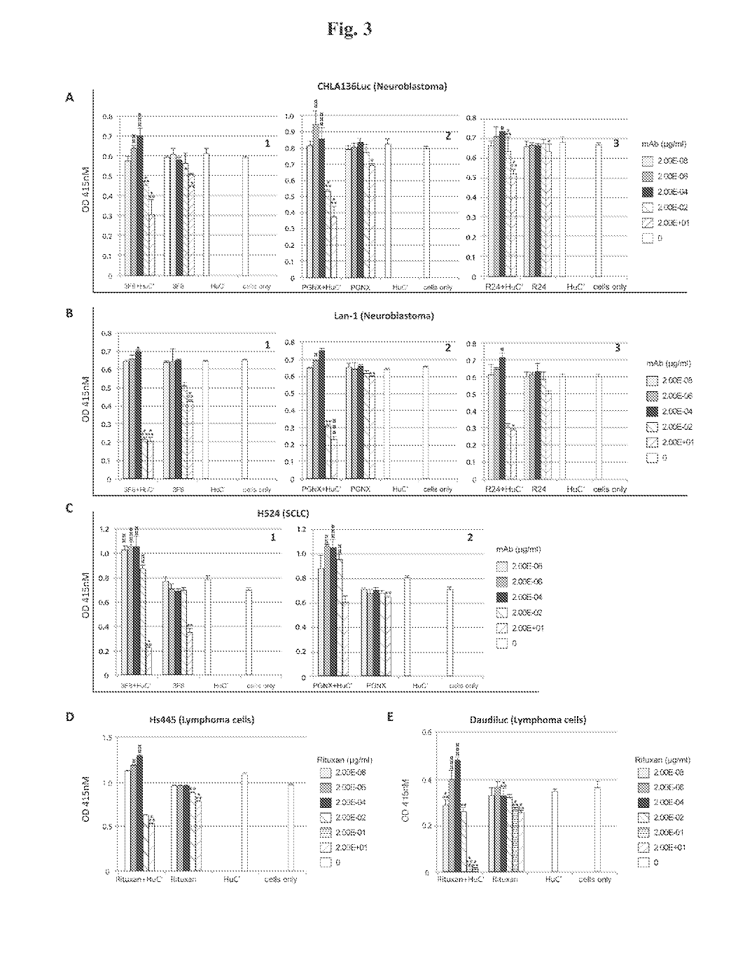

Initial experiments focused on impact of low (1 or 5 mcg or “μg”) and high (50 mcg) doses of mAbs administered weekly for 4 weeks beginning 2 days after IV challenge with 0.5×10⁶ CHLA136 cells. Survival was significantly prolonged by the 50 mcg dose of PGNX (against GM2), 3F8 (GD2), or R24 (GD3), compared with untreated mice or mice receiving low-dose PGNX (FIG. 2A), and survival was more prolonged when the 3 mAbs were administered together. While survival of mice receiving the 5 mcg dose of PGNX was not significantly changed compared with the untreated control group, tumor growth measured by luciferase expression at 6-8 weeks was significantly increased (FIG. 2B). In subsequent experiments, the 1 mcg doses of PGNX and R24 were found to be optimal for this growth enhancement at weeks 4-8 (FIGS. 2C and D). Significant enhancement of early growth was seen at low mAb doses in 5 of 6 experiments for PGNX and 2 of 2 experiments for R24. Significantly decreased survival was seen at the 1 mcg dose in 3 of 6 experiments for PGNX and 2 of 2 experiments for R24. The 1 mcg and 2 mcg doses of 3F8 and doses as low as 0.001 mcg of Rituxan resulted in slight delay of tumor growth; accelerated tumor growth was not seen at doses down to 0.02 mcg of 3F8 and 0.001 mcg of Rituxan (data not shown).

### Example 4

### Antibody Titers Resulting from High and Low Dose mAb Administration Against these Antigens

Sera drawn beginning 4 hours after administration of a high dose (50 mcg) of mAbs PGNX, 3F8, R24 demonstrated antibody titers between 1/160 and 1/1280 at 4 hours which diminished gradually over the next 2 weeks (Table 1). The 1 mcg dose of R24 and PGNX that resulted in early accelerated tumor growth in vivo resulted in minimal or no detectable antibody titers at 4 hours.

TABLE 1
Median serum titer (reciprocal) after 1 mcg or 50 mcg mAb injection*

| | 3F8 (IgG3) | | R24 (IgG3) | | PGNX (IgM) | |
|---|---|---|---|---|---|---|
| Interval | 1 mcg | 50 mcg | 1 mcg | 50 mcg | 1 mcg | 50 mcg |
| 4 h | 80 | 1280 | 20 | 640 | 0 | 160 |
| 24 h | 40 | 320 | 0 | 320 | 0 | 40 |
| 4 d | 0 | 320 | 0 | 160 | 0 | 0 |
| 7 d | 0 | 160 | 0 | 80 | 0 | 0 |
| 14 d | 0 | 80 | 0 | 80 | 0 | 0 |

*MAbs at doses indicated were injected intravenously into SCID mice. Serum was collected at intervals after the injection for determination of titer by ELISA.Titers presented here are the median for groups of 3 mice.

### Example 5

### Impact of High and Low Doses of mAbs and Complement on Tumor Cell Growth In Vitro

All 4 of the mAbs (PGNX, R24, 3F8 and Rituxan) inhibited tumor growth in vitro at high mAb doses, and accelerated tumor cell growth at low mAb doses exclusively in the presence of complement (FIG. 3). FIG. 3 represents multiple experiments with each of the cell lines. Of 7 experiments conducted on CHLA136 target cells, PGNX, 3F8 and R24 demonstrated significant low dose acceleration of growth; statistically significant for PGNX, 5 times each for 3F8 and 4 times for R24. Of 3 experiments conducted with LAN1, statistically significant growth acceleration was seen twice with each of the 3 mAbs. Five experiments were conducted on H524 with PGNX, 3F8, and R24. Significant growth acceleration was seen in 4 of these 5 experiments with each mAb. Six experiments were conducted with Hs445 and Rituxan. Low dose Rituxan significantly accelerated growth 4 time and also in a single experiment conducted on Daudi cells. In each case, high doses resulted in diminished cell counts in every experiment, which was primarily complement dependent. In each case, the low dose effects were exclusively complement dependent (FIG. 3). No acceleration of tumor growth was detected in the absence of complement, though at the highest mAb doses, complement-independent tumor inhibition was detected with 3F8 and Rituxan (FIGS. 3C-E).

The presence of bound antibody and complement activation at the CHLA136Luc cell surface was confirmed after treatments with doses of PGNX mAb as low as 0.0002 μg/ml (data not shown). Low dose PGNX (0.0002 μg/ml) bound weakly but detectably to CHLA136luc (data not shown), and terminal complement complex formation in the presence of complement (human serum) was PGNX dose-dependent and detectable down to the 0.0002 μg/ml dose level (data not shown), but was not formed when C7 depleted human serum was used as a complement source.

Overall, this complement-dependent in vitro growth inhibition at high mAb doses and acceleration of growth at low mAb doses was true with 5 different human cell lines, and included 1 IgM and 3 IgG mAbs targeting 3 glycolipid antigens (GM2, GD2 and GD3) and 1 protein antigen (CD20).

### Example 6

### Impact of Blocking PI3K/AKT on mAb Induced In Vitro Growth Inhibition and Acceleration

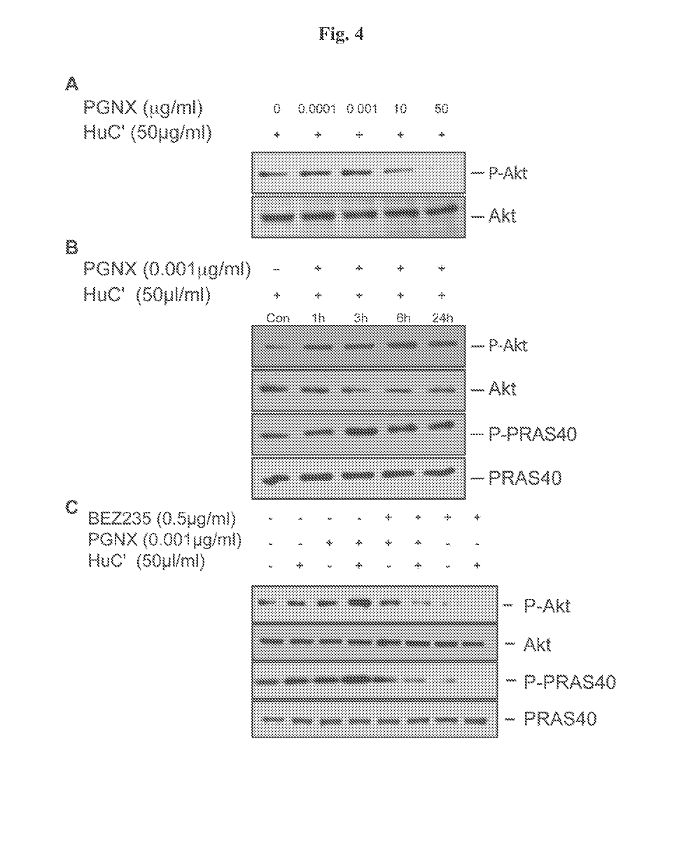

Involvement of the PI3K/Akt pathway in CHLA136luc cell growth promoted by low-dose PGNX-mediated sublytic complement activation was investigated. A PGNX level of ˜0.01 μg/ml for 4-6 hours resulted in the greatest increase in phosphorylated Akt (P-Akt) expression, while the highest doses of PGNX greatly decreased p-Akt expression (FIGS. 4A, 4B). The impact of this increased Akt activation on downstream events was tested. PRAS40 is an Akt substrate and mTORC1 inhibitory binding protein that relieves mTORC1 activity when phosphorylated. Treatment of CHLA136Luc cells with 0.001 μg/ml PGNX for 4 hours resulted in increased PRAS40 phosphorylation (FIG. 4B). The impact of mAb-mediated sublytic complement activation on PI3K/Akt/mTOR pathway activation was further demonstrated by its inhibition using the PI3K and mTOR dual inhibitor BEZ235. BEZ235 inhibited both p-Akt and p-PRAS40 expression (FIG. 4C).

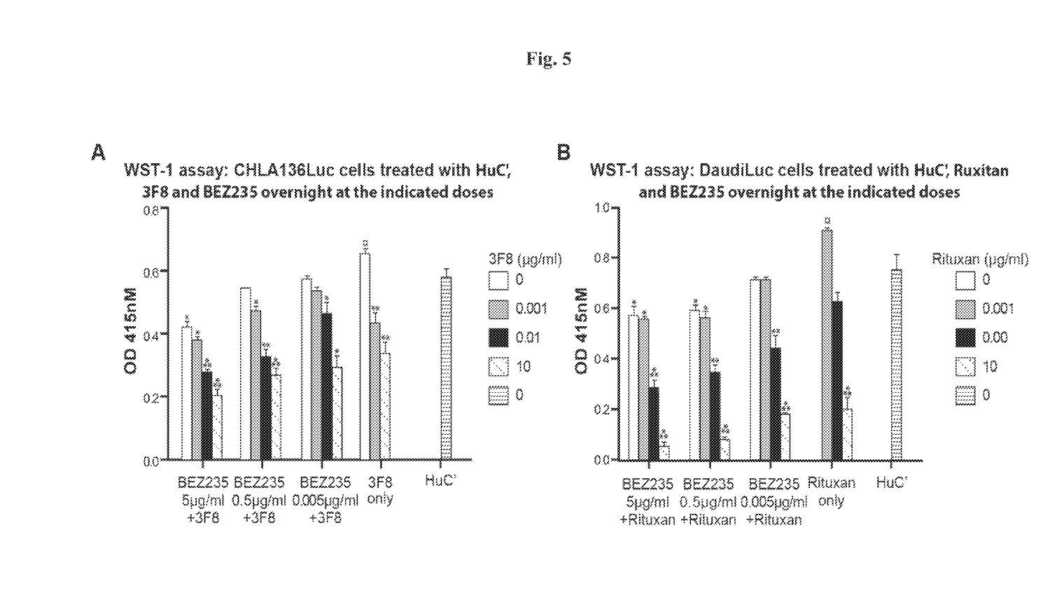

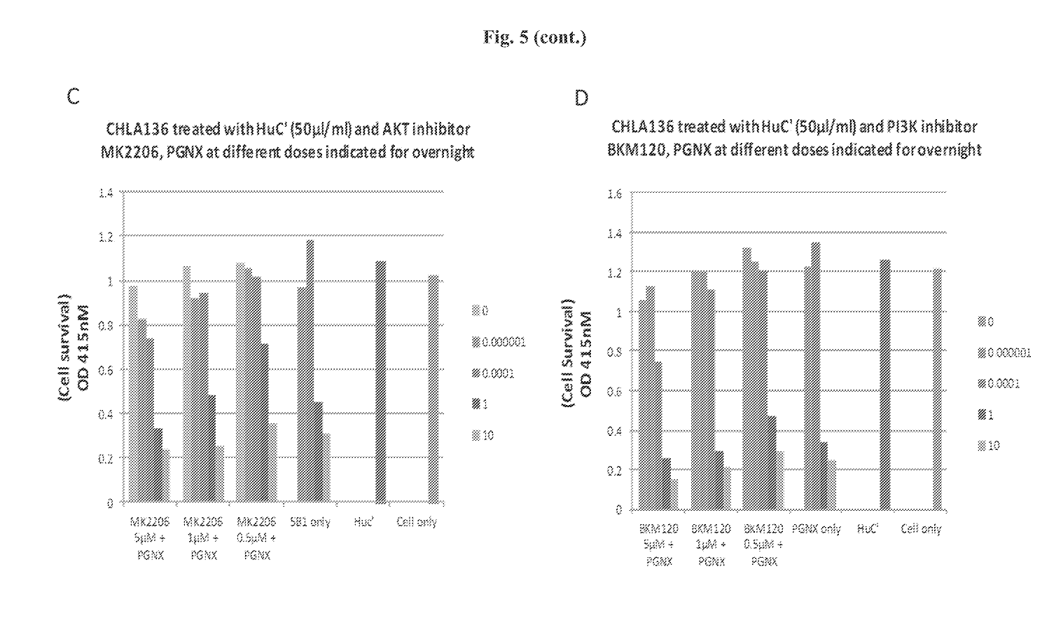

BEZ235 decreased not only PI3K/Akt/mTOR pathway activation but also CHLA136-Luc and Daudi-Luc cell growth in vitro, especially in the presence of mAbs (FIG. 5). At all doses tested, BEZ235, combined with 3F8 and Rituxan at various doses, significantly enhanced mAb cytotoxicity of CHLA136-luc and Daudiluc cells compared with each treatment alone (FIGS. 5 A, B). When combined with low-dose 3F8 (0.001 μg/ml) and Rituxan (0.0001 μg/ml), BEZ235 significantly inhibited accelerated CHLA136luc and Daudiluc cell growth induced by these low doses of mAbs (FIGS. 5 A, B). These findings were unchanged when heat-inactivated complement was used as a negative control in place of no complement. These results with 3F8 and Rituxan were consistent over several experiments with P values compared with antibodies and human complement alone ranging between 0.015 and 0.0001. Comparable results were obtained with mAbs R24 and PGNX against GD3 and GM2 (Table 2). Wortmannin (another PI3K inhibitor) also abrogated CHLA136Luc accelerated cell growth induced by low-dose 3F8, but the impact was less striking (data not shown; P values 0.04-0.008) when compared with low-dose 3F8 and human complement alone.

Treatment with specific inhibitors MK2206 (inhibitor of AKT; FIG. 5C) or BKM120 (inhibitor of PI3K; FIG. 5D) also inhibited the tumor cell (Colo205) growth in the presence of high concentration of antibody better than either inhibitor alone. When combined with low dose PGNX (e.g., 0.0001 μg/ml), both inhibitors dramatically inhibited tumor cell growth induced by low dose PGNX and complement (HuC′, 50 μl/ml). Both specific inhibitors also enhanced PGNX induced tumor cell cytotoxicity at the highest PGNX and inhibitor dose tested.

TABLE 2
Impact of treatment for 18 hours with BEZ235 at 0.5 μg/ml and increasing doses of mAbs
on growth of CHLA136 and DaudiLuc cells in WST-1 assays

| | CHLA136Luc* | | | | | | Daudiluc | |
|---|---|---|---|---|---|---|---|---|
| | mAb 3F8 | | mAb R24 | | mAb PGNX | | mAb Rituxan | |
| | % of change | | % of change | | % of change | | % of change | |
| | vs. HuC′ | P value | vs. HuC′ | P value | vs. HuC′ | P value | vs. HuC′ | P value |
| BEZ235 0.5 μg/ml alone | 6.18 ↓ | 0.200 | 18.01 ↓ | 0.027 | 28.20 ↓ | 0.016 | 21.32 ↓ | 0.000 |
| BEZ235 0.5 μg/ml + mAB0.0001 | 24.83 ↓ | 0.004 | 36.16 ↓ | 0.001 | 36.51 ↓ | 0.000 | 25.07 ↓ | 0.015 |
| BEZ235 0.5 μg/ml + mAB0.01 | 43.76 ↓ | 0.001 | 39.45 ↓ | 0.003 | 52.84 ↓ | 0.002 | 53.73 ↓ | 0.001 |
| BEZ235 0.5 μg/ml + mAB10 | 53.89 ↓ | 0.000 | 42.48 ↓ | 0.001 | 78.85 ↓ | 0.005 | 88.33 ↓ | 0.000 |
| mAb 0.0001 μg/ml | 20.48 ↑ | 0.022 | 1.64 ↑ | 0.424 | 14.93 ↑ | 0.048 | 20.48 ↑ | 0.023 |
| mAb 0.01 μg/ml | 17.45 ↓ | 0.008 | 13.34 ↑ | 0.054 | 29.46 ↑ | 0.015 | 16.15 ↓ | 0.055 |
| mAb 10 or 20 μg/ml | 52.12 ↓ | 0.002 | 19.68 ↓ | 0.004 | 60.09 ↓ | 0.000 | 72.67 ↓ | 0.001 |

* Expreiments on CHLA136 with the 3 mAbs were performed on a different dates, approximately a week apart, with PGNX first, then R24, then 3F8, for testing with the same sample of NVP-BEZ235. Some of the difference is apparent NVP-BEZ235 activity may be due to solublized BEZ235 instability.

### Example 7

### Impact of PI3K Inhibitor on mAb-Induced Accelerated Tumor Growth In Vivo

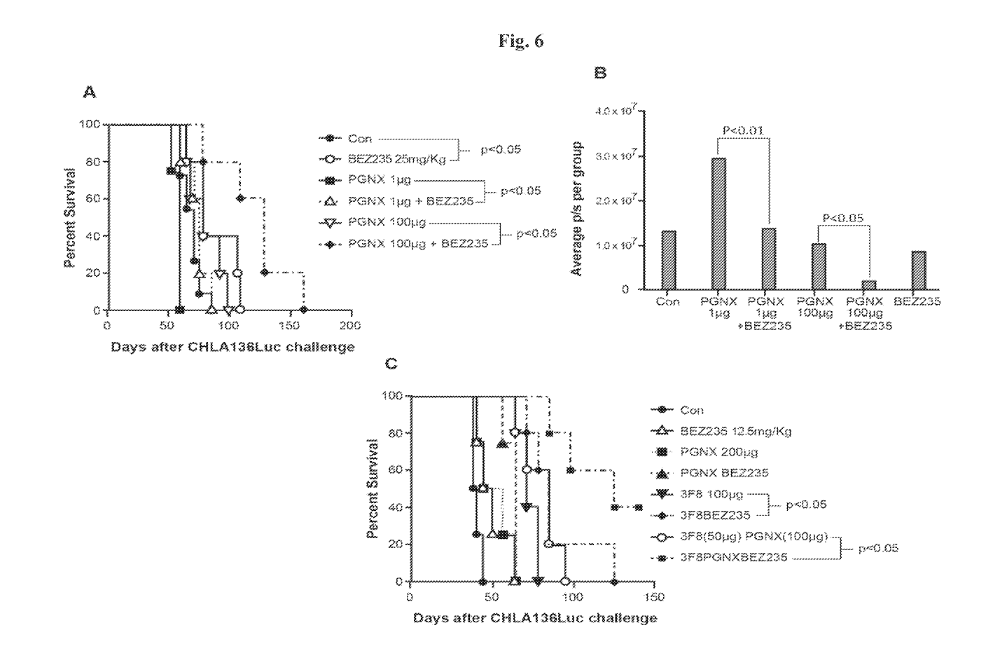

The impact of treatment with PGNX and/or 3F8 alone or in combination with BEZ235 on the growth of CHLA136Luc was tested in a SCID xenograft model (FIG. 6). Addition of BEZ235 alone significantly reduced CHLA136Luc growth and prolonged survival. The combination of BEZ235 and PGNX and/or 3F8 resulted in a further, more significant, prolongation of survival. BEZ235 also eliminated the early tumor growth acceleration induced by low-dose PGNX.

### Example 8

### Impact of PI3K Inhibitor on mAb-Induced Accelerated Tumor Growth in Colorectal Adenocarcinoma Cell Line

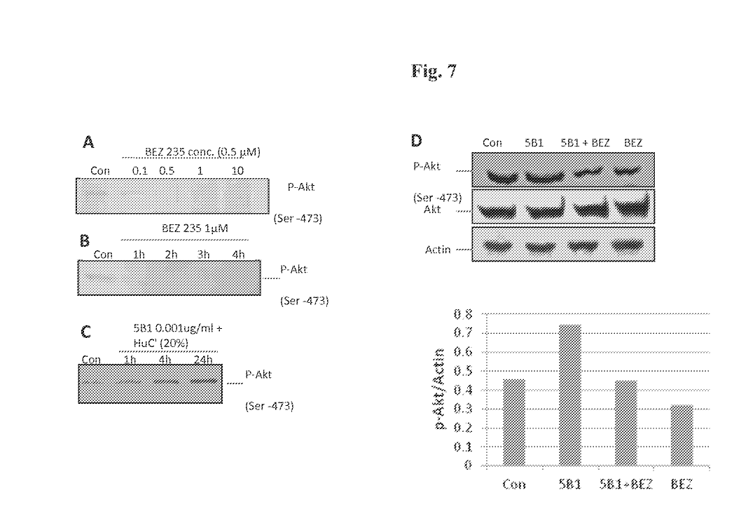

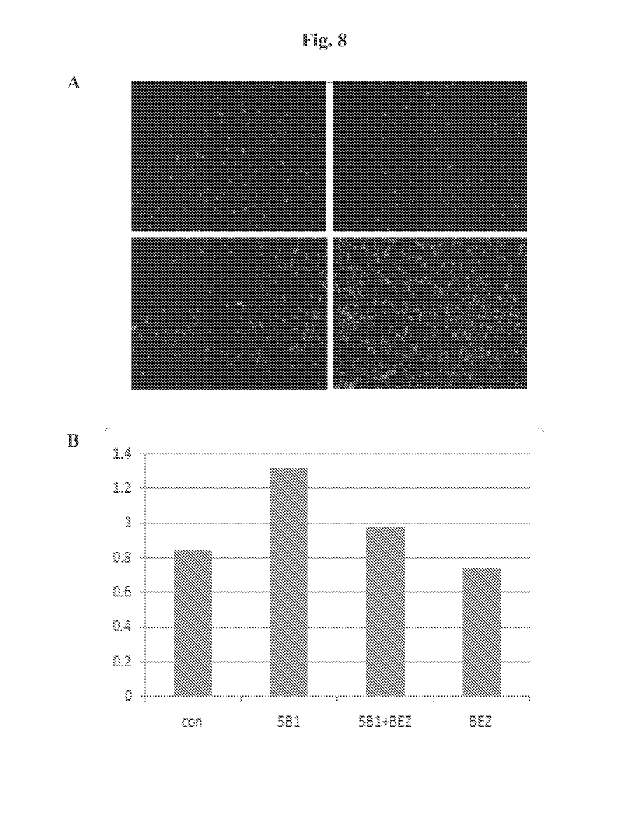

PI3K inhibitor BEZ235 was tested for its impact on the Akt activity of Colo205Luc cells alone or in combination with mAb 5B1. Both Western blots and immunohistology showed that constitutive expression of p-Akt on Colo205Luc cells was inhibited by BEZ235 (1 μM) treatment. (FIGS. 7, 8). Low dose 5B1 alone induced Akt activation and the combination of BEZ235 and 5B1 (0.001 μg/m) reduced the p-Akt expression level to background. Thus, it was demonstrated that several complement-activating mAbs against ganglioside and glycoprotein antigens exert their effects on tumor cells through modulating the PI3K/AKT pathway in the presence of complement.

Another signal transduction pathway, the Ras/MEK/Erk pathway is also frequently deregulated in human cancer as a result of genetic alterations in their components or upstream activation of cell surface receptors. Thus, additional experiments were conducted to determine whether MEK inhibition could enhance cytotoxicity of low dose, sublytic mAb treatment (i.e., overcome the pro-survival and pro-growth effects of low dose mAb treatments.

Cell growth experiments were conducted as described herein. These experiments demonstrated that the MEK inhibitor AZD6244 (0.1 μM-5.0 μM) enhanced the cytotoxicity of PGNX at sublytic low dosages (e.g., less than 0.0001 μg/m) (data not shown). Similar results were obtained with the MEK inhibitor GSK202011 (data not shown). These results suggest that the use of dual inhibitors (or a single bi-efficacious inhibitor) targeting both the PI3K and MEK/Erk pathways could enhance the efficacy of anti-cancer mAb treatments.

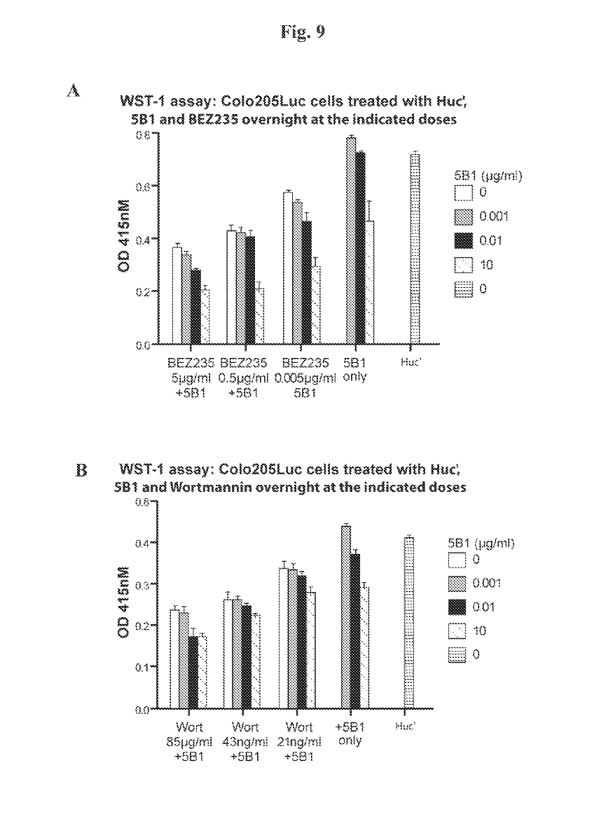

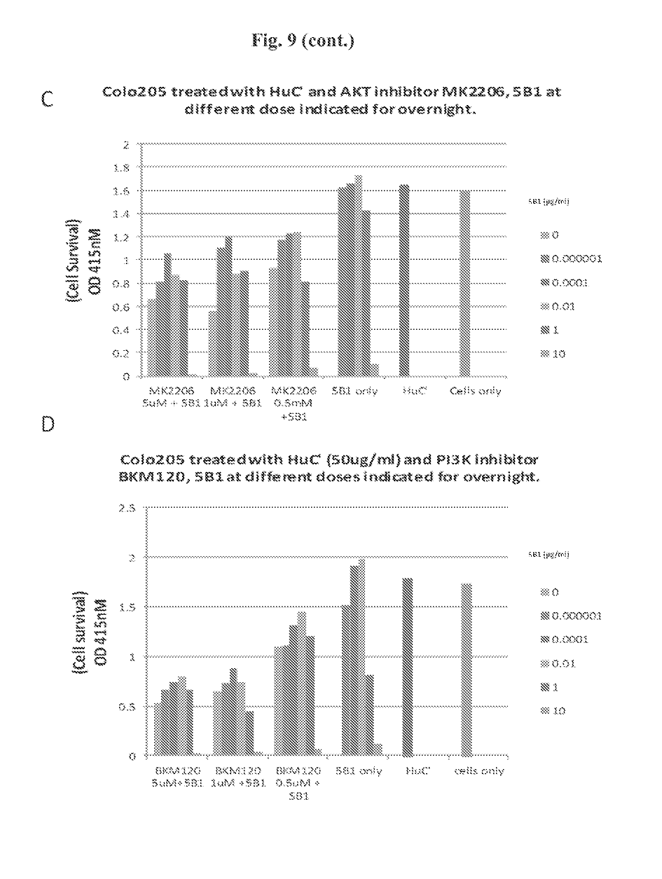

The impact of BEZ235 was compared to Wortmannin on CHLA136Luc and Colo205luc cell growth (See FIG. 9). BEZ235, compared to Wortmannin, showed a greater effect on proliferation of both CHLA136Luc and Colo205Luc cells. BEZ235 at 0.005-5.0 μg/m combined with 5B1 at 0.001 to 10 μg/m significantly enhanced 5B1 cytotoxicity of Colo205Luc cells compared to 5B1 alone, and inhibited accelerated cell growth induced by low dose 5B1 (0.001-0.01 μg/ml) (FIG. 9A). Like BEZ235, Wortmannin also abrogated Colo205Luc accelerated cell growth induced by low dose 5B1 (0.001 μg/ml) (FIG. 9B). BEZ235 at all doses tested, combined with high dose of 5B1 (20 μg/ml), significantly enhanced cytotoxicity of Colo205luc cells in a dose-dependent manner. The combination of BEZ235 with low dose 5B1 inhibited the accelerated growth induced by low dose 5B1 (0.001 μg/m) (FIG. 9A). Again, similar results were seen when Wortmannin was tested. The combination of high dose Wortmannin (85 μg/ml) with high dose 5B1 (10 μg/m) significantly enhanced tumor cell killing of Colo205Luc (FIG. 9B) and eliminated the low dose 5B1 acceleration of cell growth. MK2206 (a specific allosteric AKT inhibitor) and BKM120 (a specific inhibitor of class 1 PI3K) also enhanced the efficacy of 5B1 cytotoxicity at all doses tested (FIGS. 9C and 9D, respectively). In sum, these findings demonstrated that a variety of both general and specific inhibitors of the PI3K/Akt pathway enhanced the mAbs tumor cytotoxicity and inhibited the increased tumor growth induced by the low dose of mAbs and human complement.

### Example 9

### Discussion

The role of vaccine-induced antibodies and T cells targeting cancer antigens has been investigated. While one vaccine (Sipuleucel-T) was FDA approved for use in patients with prostate cancer, its mechanism of action remains unclear. On the other hand, several recent randomized trials with whole-cell vaccines or carbohydrate conjugate vaccines have demonstrated no clinical benefit or an initial shortened time to recurrence compared with control groups. The shortened time to recurrence seen in patients receiving the whole irradiated melanoma cell vaccine Canvaxin is difficult to dissect, since its mechanism of action (B-cell or T-cell mediated) and relevant target antigens is unclear, and any single immune response was detectable in only a minority of patients. Two of these trials targeted GM2 ganglioside using a GM2-KLH conjugate vaccine compared with interferon alpha or no treatment. This vaccine is known to induce only an antibody response and only against GM2, and to induce this response in essentially every vaccinated patient. The significantly decreased progression-free and overall survival identified during the initial 1-2 years of follow-up, though not after longer-term follow-up, in these trials is assumed to be a consequence of the vaccine-induced antibodies targeting GM2.

While GM2 is expressed on most melanomas, it is expressed in only small amounts in most cases; less than 20% of melanoma cell lines can be lysed with high doses of anti-GM2 antibodies and human complement. Consequently, it is likely that previous clinical trials with the GM2-KLH vaccine induced sublytic levels of cell surface complement activation in most cases.

It is demonstrate here that in a setting where high-dose PGNX (an IgM monoclonal antibody targeting GM2) is able to delay or prevent growth of strongly GM2 positive tumor cells both in vivo and in vitro, low (sublytic) levels of the same monoclonal antibody accelerates initial tumor growth in both settings.

Both inhibition and acceleration of tumor cell growth in vitro are shown to be complement-dependent; little or no impact on tumor growth was seen in the absence of complement. Surprisingly, these findings were not limited to the IgM mAb against GM2. The same complement-dependent, high-dose inhibition of tumor growth and low-dose acceleration of tumor growth in vitro was seen with IgG mAbs targeting glycolipid antigens GD2, GD3, glycoprotein (and glycolipid) antigen sialyl Lewis A, and the protein antigen CD20 (using Rituxan) on 5 different cell lines. Inhibition or prevention of tumor growth at high mAb doses and early acceleration of tumor growth at low levels was seen in vivo as well in a SCID mouse model, with monoclonal antibodies targeting not only GM2 but also GD3 and sialyl Lewis A. The high dose (50 mcg) of these mAbs is comparable to doses of mAbs commonly used in patients on a per Kg basis and results in antibody titers in mice at 4 and 24 hours in the 1/160-1/1280 range. The low dose (0.01-1 mcg) resulted in little or no detectable serum antibody even at 4 hours.

Long-lasting antibody titers in the range of 1/320-1/1280 against these same antigens are induced in most patients by KLH conjugate vaccines. This suggests that if used in the setting of high-antigen-expressing tumors, the monovalent vaccines should be beneficial, not detrimental, and that polyvalent vaccines inducing antibody titer against several cell surface antigens should be even more beneficial

No previous studies exploring sublytic complement activation have involved tumor cells, and no others have involved mAbs or immune sera targeting cancer antigens. It has been shown herein that high doses of antibodies against each of the glycolipid or glycoprotein antigens and one protein antigen that we tested all decreased tumor cell growth in vitro in the presence of human complement while low doses of each increased tumor growth.

Sublytic complement activation at the cell surface can activate a variety of metabolic processes resulting in adherence, aggregation, mitogenesis, and proliferation of a variety of nonmalignant cell types. Enhanced HIV infection, glomerular mesangial cell proliferation associated with glomerulonephritis, and protection from subsequent lytic complement doses have been demonstrated as consequences. Several signal transduction pathways may be responsible for the cell-cycle activation, anti-apoptotic, and differentiation properties associated with sublytic complement levels. These include primarily activation of the PI3K/Akt pathway. Involvement of the PI3K/Akt signaling pathway in accelerated tumor growth induced by sublytic levels of antibody-mediated complement activation has not previously been explored.

It is demonstrated here that the accelerated cell growth induced by treatment with low-dose mAbs was associated with activation of the PI3K/Akt/mTOR pathway. Treatment with low-dose PGNX (0.001 μg/m) and human complement induced increased Akt phosphorylation, and also increased release of the phosphorylated Akt substrate PRAS40, a raptor binding protein that inhibits mTORC1 kinase activity. These data demonstrate involvement of the PI3K/AKT/mTOR pathway in low-dose mAb sublytic complement activation induced accelerated CHLA136Luc cell growth.

Testing with inhibitors of this pathway supported this. BEZ235 is a dual-PI3K and mTOR inhibitor, inhibiting both the catalytic subunit (P110) of PI3K and mTORC, while Wortmannin is a more specific PI3K inhibitor, binding to the P110 catalytic subunit of PI3K. It was demonstrated here that constitutive expression and low-dose mAb-induced increased expression of p-Akt and p-PRAS40 in CHLA136Luc cells was inhibited by BEZ235. BEZ235 and Wortmannin also significantly enhanced in vitro tumor cytotoxicity with high-dose 3F8, 5B1, R24, PGNX and Rituxan mAbs in a dose-dependent manner, and inhibited in vitro tumor growth acceleration induced by low doses of these same mAbs. BEZ235 also increased the efficacy of mAbs PGNX and 3F8 against CHLA136luc cells in vivo, significantly increasing survival of challenged SCID mice compared with high-dose PGNX and 3F8 alone, and preventing the early tumor growth acceleration seen with low dose PGNX.

It has also been demonstrated that MEK inhibitors (e.g., AZD6244 and GSK202011) enhanced the cytotoxicity of mAbs (e.g., PGNX) at sublytic low dosages (e.g., less than 0.0001 μg/ml). These results suggest that the use of dual inhibitors (or a single bi-efficacious inhibitor) targeting both the PI3K and MEK/Erk pathways could enhance the efficacy of anti-cancer mAb treatments. Without wishing to be bound by any particular theory, it is possible that inhibit of only one pathway (e.g., PI3K) could sometimes cause compensatory activation of another survival pathway (e.g., MEK/Erk). It has also been demonstrated that a variety of both general and specific inhibitors of the PI3K/Akt pathway enhanced the mAbs tumor cytotoxicity and inhibited the increased tumor growth induced by the low dose of mAbs and human complement.

In summary, complement-activating antibodies are a two-edged sword, demonstrating potent antitumor activity at high (clinically relevant) doses and weak tumor enhancing or accelerating activity at very low doses. Therapy with complement-activating antibodies should be restricted to treatment of antigen-rich tumors. Sublytic complement activation, which can result from a low level of antibody or low antigen expression, results in increased activation of the PI3K/Akt survival pathway and accelerated tumor growth. This can be eliminated by treatment with PI3K inhibitors (e.g., BEZ235, Wortmannin, MK2206 and BKM120), which also increase the efficacy of even high doses of these mAbs. Furthermore, manipulation of the PI3K/Akt pathway and its signaling network can potentially increase the potency of passively administered mAbs and vaccine-induced antibodies targeting a variety of tumor cell surface antigens.

### Example 10

### The Effects of Specific PI3K/Akt/mTOR Pathway Inhibitors on In Vitro Cytotoxicity

The ability of specific PI3K/Akt/mTOR pathway inhibitors on in vitro cytotoxicity of sublytic and lytic complement activation is determined as above. Specifically, cell growth of CHLA136Luc, Lan-1, H524, HS445, DaudiLuc and Colo205Luc cells is promoted by low-dose 3F8-, R24-, PGNX- and Rituxan-mediated sublytic complement activation. mAb levels of ˜0.0001-0.01 μg/ml for 4-6 hours result in activation of the PI3K/Akt/mTOR pathway and increases phosphorylated Akt (P-Aid) expression. The therapeutic potential of inhibition of this pathway is evaluated using the PI3K-specific inhibitors BKM120 and LY49002, the Akt inhibitor MK2206, and the mTOR inhibitor Rapamycin. Different doses of inhibitors are evaluated in combination with different mAbs.

These inhibitors decrease not only PI3K/Akt/mTOR pathway activation but also cell growth in vitro in the presence of mAbs. At all doses and combinations tested, the concurrent administration mAbs and inhibitors significantly enhances mAb cytotoxicity of the cells compared with each treatment alone and significantly inhibits accelerated cell growth induced by these low doses of mAbs.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a”, “an” and “the” may mean one or more than one unless indicated to the contrary or otherwise evident from the context. Thus, for example, reference to “an antibody” includes a plurality of such antibodies, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are presenting, employed in, or otherwise relevant to a given product or process. Furthermore, it is to be understood that the invention encompasses all variations, combinations, and permutations in which one or more limitation, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for anyone of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understand of one of ordinary skill in the art, values that are expressed as ranges can assume any specific value or sub-range within the state ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

We claim:

1. A method of potentiating an antibody-based cancer treatment, the method comprising
administering to a subject a therapeutically effective amount of at least one complement-mediating antibody against a cancer antigen or a cancer vaccine capable of inducing antibodies against the cancer antigen; and
concurrently administering to the subject at least one PI3K inhibitor that inhibits one or more components of the PI3K pathway.

2. The method of claim 1, wherein the cancer antigen is selected from the group consisting of GM2, GD2, GD3, fucosyl GM1, Neu5Gc, CD20, Lewis Y, sialyl Lewis A, Globo H, Thomsen-Friedenreich antigen, Tn, sialylated Tn, Mucin 1, adenocarcinoma-associated antigen, prostate-specific antigen, polysialic acid, and CA125.

3. The method of claim 1, wherein the complement-mediating antibody is selected from a group consisting of alemtuzumab, bevacizumab, cetuximab, panitumumab, rituximab, pertuzumab, tositumomab, gemtuzumab ozogamicin, and combinations thereof.

4. The method of claim 1, wherein the PI3K inhibitor inhibits Akt1, Akt 2 or Akt3.

5. The method of claim 1, wherein the PI3K inhibitor inhibits p110.

6. The method of claim 5, wherein the PI3K inhibitor inhibits p110α.

7. The method of claim 1, wherein the PI3K inhibitor inhibits mTOR.

8. The method of claim 7, wherein the PI3K inhibitor is BEZ235.

9. The method of claim 1, wherein the PI3K inhibitor is selected from a group consisting of Wortmannin, F-1126, BEZ-35, BKM120, BYL719, XL-147, GDC-0941, BGT226, GSK1059615, GSK690693, XL-765, PX866, GDC0941, CAL101, Perifosine, VQD002, MK2206, and combinations thereof.

10. The method of claim 1, further comprising concurrent administration of at least one MEK inhibitor.

11. The method of claim 1, wherein the therapeutically effective amount of complement-mediating antibody comprises at least one dose of about 1-150 milligrams per kilogram (kg) of body weight of the subject.

12. The method of claim 2, wherein the step of administering an anti-tumor antibody comprises administering at least one dose of about 40-50 milligrams per kilogram of body weight to the subject.

13. The method of claim 1, wherein the PI3K inhibitor is orally or parenterally administered in an amount sufficient to deliver from about 1-150 milligram per kilogram (kg) of body weight of the subject.

14. The method of claim 1, wherein the antibody-based cancer treatment is used for treating a neuroblastoma, lymphoma, colon cancer, breast cancer, sarcoma, melanoma, pancreatic cancer, prostate cancer, ovarian cancer or small lung carcinoma.

15. The method of claim 1, further comprising determining a level of expression of the tumor cell surface antigen.

16. The method of claim 1, further comprising concurrent administration of an anti-cancer treatment.

17. The method of claim 16, wherein the anti-cancer treatment is selected from the group consisting of cytotoxic agents, radiation, and surgery.

18. The method of claim 17, wherein the cytotoxic agents are selected from the group consisting of cisplatin, carboplatin, doxorubicin, etoposide, cyclophosphamide, methotrexate, taxol, gemcitabine and celecoxib.

19. A method of administering cancer vaccine to a subject, the method comprising concurrently administering a PI3K inhibitor to the subject.

20. The method of claim 19, wherein the cancer vaccine is a polyvalent vaccine.

21. The method of claim 19, wherein the cancer vaccine is a monovalent vaccine.

22. The method of claim 19, wherein the cancer vaccine induces complement-mediating antibodies against a cell surface protein selected from the group consisting of a carbohydrate epitope, a glycolipid epitope, a glycoprotein epitope or a mucin.

23. The method of claim 19, wherein the carbohydrate epitope is selected from the group consisting of GM2, GD2, GD3, fucosyl GM1, Neu5Gc, CD20, Lewis Y, sialyl Lewis A, Globo H, Thomsen-Friedenreich antigen, Tn, sialylated Tn, Mucin 1, adenocarcinoma-associated antigen, prostate-specific antigen, polysialic acid, CA125, and unimolecular multiantigenic constructs comprising a STn cluster, TN cluster and/or TF clustered antigens.

24. The method of claim 19, wherein the cancer vaccine comprises an antigen chemically conjugated to a carrier molecule.

25. The method of claim 14, wherein the carrier molecule is selected from the group comprising keyhole limpet hemocyanin, Neisseria meningitidis outer membrane proteins, multiple antigenic peptide, cationized bovine serum albumin and polylysine.

26. The method of claim 19, wherein the cancer vaccine further comprises an adjuvant.

27. The method of claim 27, wherein the adjuvant is selected from the group comprising CRL-1005 (polypropylene), CpG ODN 1826 (synthetic bacterial nucleotide), GM-CSF (peptide), MPL-SE (monophosphoryl lipid A), GPI-0100 (hydrolyzed saponin fractions), MoGM-CSF (F_{c}-GM-CSF fusion protein), PG-026 (Peptidoglycan), QS-21 (saponin fraction), synthetic QS-21 analogs, TiterMax Gold (CRL-8300 (polyoxypropylene; polyoxyethylene), and analogs thereof.

28. A method for identifying subjects suitable for treatment with complement-mediating anti-tumor antibodies, the method comprising:
quantifying in a sample from a subject suffering from or susceptible to cancer an expression level of an antigen that is differentially expressed in cancer cells relative to normal cells, which antigen is recognized by at least one antibody that activates complement; and
determining that the expression level is above or below a threshold correlated with responsiveness to complement-activating therapy.
